(19) United States
(12) Patent Application Publication
(10) Pub. No.: US 2022/0227696 A1
(43) Pub. Date: Jul. 21, 2022

(54) Multifunctional Fluorinated Compound, Fluorinated Polymers Made from the Compound, and Related Methods

(71) Applicant: 3M INNOVATIVE PROPERTIES COMPANY, St. Paul, MN (US)

(72) Inventors: Markus E. Hirschberg, Mühldorf (DE); Klaus Hintzer, Kastl (DE); Gerd-Volker Röschenthaler, Bremen (DE); Sergey N. Tverdomed, Bremen (DE); Romana Pajkert, Bremen (DE)

(21) Appl. No.: 17/614,608

(22) PCT Filed: Jun. 2, 2020
(86) PCT No.: PCT/IB2020/055202

§ 371 Date: Nov. 29, 2021

Related U.S. Application Data
(60) Provisional application No. 62/856,893, filed on Jun. 4, 2019.

(51) Int. Cl. C07C 69/65; C08F 214/18; C07C 67/347; C07C 57/52; C07C 51/09; C07F 7/18; C07C 57/76; C07C 51/60

(52) U.S. Cl. CPC C07C 69/65; C08F 214/18; C07C 67/347; C07C 51/60; C07C 51/09; C07F 7/1896; C07C 57/76; C07C 57/52

(57) ABSTRACT

The multifunctional compound is represented by formula X—C(R)RF—Y, in which X and Y are each independently —C(O)—O-M, —C(O)-HAL, —C(O)—NR¹₂, —C≡N, —C(O)NR¹—SO₂—R_{f}¹-W, or a fluorinated alkenyl group that is optionally interrupted by one or more —O— groups. HAL is —F, —Cl, or —Br. R_{f}¹ is a fluorinated alkylene group that is optionally interrupted by one or more —O— groups. W is —F, —SO₂Z, —CF═CF₂, —O—CF═CF₂, or —O—CF₂—CF═CF₂. Z is —F, —Cl, —NR¹₂, or —OM. Each R¹ is a hydrogen atom or alkyl having up to four carbon atoms. M is an alkyl group, a trimethylsilyl group, a hydrogen atom, a metallic cation, or a quaternary ammonium cation. R is a bromine, chloride, fluorine or hydrogen atom; and RF is a fluorinated alkenyl group that is unsubstituted or substituted by at least one chlorine atom, aryl group, or a combination thereof or RF is a fluorinated alkyl group or arylalkylenyl group that is substituted by bromine or iodine and uninterrupted or interrupted by at least one —O— group. A process for making the compound is also disclosed. A fluoropolymer made from the compound and a method of making the fluoropolymer are also disclosed.

### CROSS-REFERENCE TO RELATED APPLICATION

This application claims priority to U.S. Provisional Application No. 62/856,893, filed Jun. 4, 2019, the disclosure of which is incorporated by reference in its entirety herein.

### BACKGROUND

Fluorinated polymers are widely used as raw materials and known for a variety of beneficial properties. Melt processable copolymers of tetrafluoroethylene (TFE) and one or more other monomers have useful properties such as chemical resistance, weather resistance, low flammability, thermal stability, and advantageous electrical properties. Such properties render these fluoropolymers useful, for example, in articles such as tubes, pipes, foils, films, and coatings for wires and cables.

Fluoroelastomers are known to have excellent mechanical properties, heat resistance, weather resistance, and chemical resistance, for example. Such beneficial properties render fluoroelastomers useful, for example, as O-rings, seals, hoses, skid materials, and coatings (e.g., metal gasket coating for automobiles) that may be exposed to elevated temperatures or corrosive environments. Fluoroelastomers have been found useful in the automotive, chemical processing, semiconductor, aerospace, and petroleum industries, among others.

Fluoroelastomers are typically prepared by combining an amorphous fluoropolymer, sometimes referred to as a fluoroelastomer gum, with one or more curatives, shaping the resulting curable composition into a desired shape, and curing the curable composition. The amorphous fluoropolymer often includes a cure site, which is a functional group incorporated into the amorphous fluoropolymer backbone capable of reacting with a certain curative.

Copolymers of TFE and monomers including sulfonyl and carboxylic pendant groups are useful as ionic copolymers or ionomers. Ionomers can be useful, for example, for making polymer electrolyte membranes for membrane electrode assemblies in fuel cells or NaCl electrolysis. Decreasing the sulfonyl group equivalent weight of the copolymer tends to increase electrical conductivity in the copolymer. U.S. Pat. No. 8,097,383 (Kaneko) reports to provide a polymer electrolyte material having a high ion exchange capacity and a low resistance and having a higher softening temperature than a conventional electrolyte material. U.S. Pat. No. 7,297,815 (Murata) reports to provide a process to obtain fluorinated sulfonyl fluoride compound having various molecular structures efficiently at a low cost.

Fluorinated polymers are typically prepared by aqueous emulsion polymerization, in which polymerization is carried out in an aqueous phase, typically in the presence of a fluorinated emulsifier. It can be desirable in some applications to remove the emulsifier from the fluorinated polymer or otherwise avoid the presence of the emulsifier in the final article.

### SUMMARY

The present disclosure provides a multifunctional fluorinated compound and a process for making it. Readily obtainable malonate esters and related carboxylic acids and their derivatives are advantageously used as starting materials for the process. The process, which includes reaction with compounds having a fluorinated alkene group can be carried out at relatively low temperature and ambient pressure. The multifunctional fluorinated compound can be useful, for example, for introducing cure sites into fluoropolymers, as polymerization aids, and in the preparation of fluorinated ionomers.

In one aspect, the present disclosure provides a multifunctional compound represented by formula:

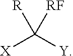

In this formula R is a fluorine, chlorine, bromine or hydrogen atom; RF is a fluorinated alkenyl group that is uninterrupted or interrupted by at least one —O— group and unsubstituted or substituted by at least one chlorine atom, aryl group, or a combination thereof or RF is a fluorinated alkyl group or arylalkylenyl group that is substituted by bromine or iodine and uninterrupted or interrupted by at least one —O— group; and X and Y are each independently —C(O)—O-M, —C(O)-HAL, —C(O)—NR¹₂, —C≡N, —C(O)NR¹—SO₂—R_{f}¹-W, or a fluorinated alkenyl group that is uninterrupted or interrupted by one or more —O— groups. Each HAL is independently —F, —Cl, or —Br. Each R_{f}¹ is independently a fluorinated alkylene group that is uninterrupted or interrupted by one or more —O— groups. Each W is independently —F, —SO₂Z, —CF═CF₂, —O—CF═CF₂, or —O—CF₂—CF═CF₂. Each Z is independently —F, —Cl, —R¹₂, or —OM. Each R¹ is independently a hydrogen atom or alkyl having up to four carbon atoms, and each M is independently an alkyl group, a trimethylsilyl group, a hydrogen atom, a metallic cation, or a quaternary ammonium cation.

In another aspect, the present disclosure provides a fluoropolymer prepared from components including the multifunctional compound.

In another aspect, the present disclosure provides a process for making the multifunctional compound. The process includes combining first components that include a malonate represented by formula M′O(O)C—C(R)H—C(O)OM′, a base, and a fluorinated compound comprising an alkene group and forming a compound represented by formula M′O(O)C—C(R)RF—C(O)OM′. In these formulas each M′ is independently an alkyl group or a trimethylsilyl group, R is a bromine, chlorine, fluorine, or hydrogen atom, and RF is fluorinated alkenyl that is uninterrupted or interrupted by at least one —O— group and unsubstituted or substituted by at least one chlorine atom, aryl group, or combination thereof.

Further process steps are useful for making the various embodiments of the multifunctional compound.

In another aspect, the present disclosure provides a method of making a fluoropolymer. The method includes combining fourth components comprising the multifunctional compound disclosed herein and at least one fluorinated monomer represented by formula R^{a}CF═CR^{a}₂, CF₂═CF(CF₂)ₘ(OCₙF₂ₙ)_{z}OR_{f}², or a combination thereof, and copolymerizing the fluorinated monomer and the multifunctional compound. In these formulas, each R^{a} is independently fluoro, chloro, bromo, hydrogen, a fluoroalkyl group, alkyl having up to 10 carbon atoms, alkoxy having up to 8 carbon atoms, or aryl having up to 8 carbon atoms; R_{f}² is a linear or branched perfluoroalkyl group having from 1 to 8 carbon atoms and optionally interrupted by one or more —O— groups; z is 0, 1, or 2; each n is independently 1, 2, 3, or 4; and m is 0 or 1.

In this application:

Terms such as “a”, “an” and “the” are not intended to refer to only a singular entity but include the general class of which a specific example may be used for illustration. The terms “a”, “an”, and “the” are used interchangeably with the term “at least one”.

The phrase “comprises at least one of” followed by a list refers to comprising any one of the items in the list and any combination of two or more items in the list. The phrase “at least one of” followed by a list refers to any one of the items in the list or any combination of two or more items in the list.

“Alkyl group” and the prefix “alk-” are inclusive of both straight chain and branched chain groups and of cyclic groups. Unless otherwise specified, alkyl groups herein have up to 20 carbon atoms. Cyclic groups can be monocyclic or polycyclic and, in some embodiments, have from 3 to 10 ring carbon atoms.

The terms “aryl” and “arylene” as used herein include carbocyclic aromatic rings or ring systems, for example, having 1, 2, or 3 rings and optionally containing at least one heteroatom (e.g., O, S, or N) in the ring optionally substituted by up to five substituents including one or more alkyl groups having up to 4 carbon atoms (e.g., methyl or ethyl), alkoxy having up to 4 carbon atoms, halo (i.e., fluoro, chloro, bromo or iodo), hydroxy, or nitro groups. Examples of aryl groups include phenyl, naphthyl, biphenyl, fluorenyl as well as furyl, thienyl, pyridyl, quinolinyl, isoquinolinyl, indolyl, isoindolyl, triazolyl, pyrrolyl, tetrazolyl, imidazolyl, pyrazolyl, oxazolyl, and thiazolyl.

“Alkylene” is the multivalent (e.g., divalent or trivalent) form of the “alkyl” groups defined above. “Arylene” is the multivalent (e.g., divalent or trivalent) form of the “aryl” groups defined above.

“Arylalkylene” refers to an “alkylene” moiety to which an aryl group is attached. “Alkylarylene” refers to an “arylene” moiety to which an alkyl group is attached.

The term “fluorinated” refers to groups in which at least some C—H bonds are replaced by C—F bonds.

The terms “perfluoro” and “perfluorinated” refer to groups in which all C—H bonds are replaced by C—F bonds.

The term multifunctional refers to having more than one functional group on the polyfluoroalkyl or perfluoroalkyl backbone. In some embodiments, multifunctional refers to trifunctional. Useful functional groups include carboxylic acids and their derivatives, sulfonic acids and their derivatives, vinyl ethers, allyl ethers, cyano groups, alkenes, amides, and halogens such as iodine and bromine. In a multifunctional (e.g., trifunctional) group, the multiple functional groups need not be the same.

The phrase “interrupted by at least one —O— group”, for example, with regard to a perfluoroalkyl or perfluoroalkylene group refers to having part of the perfluoroalkyl or perfluoroalkylene on both sides of the —O— group. For example, —CF₂CF₂—O—CF₂—CF₂— is a perfluoroalkylene group interrupted by an —O—.

All numerical ranges are inclusive of their endpoints and nonintegral values between the endpoints unless otherwise stated (e.g., 1 to 5 includes 1, 1.5, 2, 2.75, 3, 3.80, 4, 5, etc.).

### DETAILED DESCRIPTION

The present disclosure provides a multifunctional compound represented by formula I:

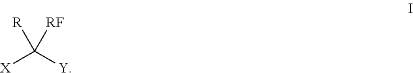

In formula I, X and Y are each independently —C(O)—O-M, —C(O)-HAL, —C(O)—NR¹₂, —C≡N, —C(O)NR¹—SO₂—R_{f}¹-W, or a fluorinated alkenyl group that is uninterrupted or interrupted by at least one —O— group.

For —C(O)—O-M, each M is independently an alkyl group, a trimethylsilyl group, a hydrogen atom, a metallic cation, or a quaternary ammonium cation. The quaternary ammonium cation can be substituted with any combination of hydrogen and alkyl groups, in some embodiments, alkyl groups independently having from one to four carbon atoms. In some embodiments, each M is independently an alkyl group, a trimethylsilyl group, a hydrogen atom, or a metallic cation. In some embodiments, each M is independently an alkyl group or a hydrogen atom. In some embodiments, each M is independently an alkyl group. In any of these embodiments, alkyl can have up to 4, 3, 2, or 1 carbon atoms. In some embodiments, M is the metallic cation, and the metallic cation is an alkali-metal cation (e.g., lithium, sodium, potassium, or cesium). In some embodiments, M is a potassium, sodium, or lithium cation.

For —C(O)-HAL, each HAL is independently —F, —Cl, or —Br. In some embodiments, each HAL is independently —F or —Cl. In some embodiments, each HAL is —F. In some embodiments, at least one of X or Y is —C(O)—F. In some embodiments, both X and Y are —C(O)—F.

In —C(O)—NR¹₂, each R¹ is hydrogen or alkyl having up to four carbon atoms (e.g., methyl, ethyl, propyl, or butyl). In some embodiments, each R¹ is hydrogen or methyl. In some embodiments, each R¹ is hydrogen.

In some embodiments, each fluorinated alkenyl group independently has up to 20, 10, 8, 6, or 4 carbon atoms. Fluorinated alkenyl groups have at least two carbon atoms, in some embodiments, at least 3 carbon atoms. Each fluorinated alkenyl group may independently be partially fluorinated or perfluorinated, may have more than one alkene group, and may have up to 6, 5, 4, 3, or 2 —O— groups. Partially fluorinated alkenyl groups can include at least one carbon-hydrogen bond, for example. In some embodiments, each fluorinated alkenyl group is independently interrupted by one —O— group. In some embodiments, each fluorinated alkenyl is independently —CF₂—O-perfluorinated alkenyl. In some embodiments, each fluorinated alkenyl group is independently —CF₂—O-perfluorinated (C₂-C₆)alkenyl. In some embodiments, each fluorinated alkenyl group is independently —CF₂—O—CF₂—CF═CF₂ or —CF₂—O—CF═CF₂. In some embodiments, the fluorinated alkenyl is not interrupted by —O— groups, and includes only carbon-carbon bonds, carbon-fluorine bonds, and optionally carbon-hydrogen or carbon-chlorine bonds. In some embodiments, both X and Y are the same fluorinated alkenyl group that is uninterrupted or interrupted by one or more —O— groups. In some embodiments, at least one of X or Y is —CF₂—O—CF₂—CF═CF₂, or —CF₂—O—CF═CF₂. In some embodiments, both X and Y are —CF₂—O—CF₂—CF═CF₂, or —CF₂—O—CF═CF₂.

For, —C(O)NR¹—SO₂—R_{f}¹-W and, each R_{f} is independently a fluorinated alkylene group that is uninterrupted or interrupted by one or more —O— groups group, each W is independently —F, —SO₂Z, —CF═CF₂, —O—CF═CF₂, or —O—CF₂—CF═CF₂, each Z is independently —F, —Cl, —NR¹₂, or —OM, each R¹ is independently hydrogen or alkyl, and wherein each M is independently an alkyl group, a trimethylsilyl group, a hydrogen atom, a metallic cation, or a quaternary ammonium cation. The quaternary ammonium cation can be substituted with any combination of hydrogen and alkyl groups, in some embodiments, alkyl groups independently having from one to four carbon atoms. In some embodiments, each R¹ is hydrogen or alkyl having up to four carbon atoms (e.g., methyl). In some embodiments, each R¹ is hydrogen. In some embodiments, each M is independently an alkyl group, a hydrogen atom, or a metallic cation. In some embodiments, each M is independently an alkyl group or a hydrogen atom. In some embodiments, each M is independently an alkyl group. In any of these embodiments, alkyl can have up to 4, 3, 2, or 1 carbon atoms. In some embodiments, the metallic cation is an alkali-metal cation (e.g., lithium, sodium, potassium, or cesium). In some embodiments, M is a potassium, sodium, or lithium cation. In some embodiments, each R_{f} independently has up to 20, 10, 8, 6, or 4 carbon atoms. In some embodiments, each R_{f} has at least two carbon atoms, in some embodiments, at least 3 carbon atoms. Each R_{f}¹ may independently be partially fluorinated or perfluorinated and may have up to 6, 5, 4, 3, or 2 —O— groups. Partially fluorinated alkylene groups can include at least one carbon-hydrogen bond, for example. In some embodiments, W is —SO₂Z, —CF═CF₂, —O—CF═CF₂, or —O—CF₂—CF═CF₂. In some embodiments, W is —SO₂Z. In some embodiments, each Z is independently —F, —Cl, or —OM. In some embodiments, each Z is independently —F or —OM, wherein M is a hydrogen atom or a metallic cation.

In some embodiments, X and Y are each independently —C(O)—O-M, —C(O)F, —C≡N, or —CF₂—O-perfluorinated alkenyl, and wherein each M is independently an alkyl group, a trimethylsilyl group, a hydrogen atom, a metallic cation, or a quaternary ammonium cation. In some embodiments, at least one of X or Y is —C(O)—F, and at least one of X or Y is —CF₂—O-perfluorinated alkenyl. In some embodiments, each X and Y is independently —C(O)—O-M, each M is independently a hydrogen atom, a metallic cation, or a quaternary ammonium cation. In some embodiments, each X and Y is independently —C(O)—O-M, each M is independently an alkyl group or a trimethylsilyl group. In some embodiments, X and Y are each independently —C≡N or —CF₂—O-perfluorinated alkenyl. In some embodiments, at least one of X or Y is —CF₂—O—CF₂—CF═CF₂, or —CF₂—O—CF═CF₂. In some embodiments, both X and Y are —CF₂—O—CF₂—CF═CF₂, or —CF₂—O—CF═CF₂. In some embodiments, at least one of X or Y is —C(O)NR¹₂. In some embodiments, at least one of X or Y is —C(O)NR¹₂ and at least one of X or Y is —C(O)NR¹₂ or —C(O)NR¹—SO₂—R_{f}¹-W. In some embodiments, at least one of X or Y is —C(O)NR¹—SO₂—R_{f}¹-W. In some embodiments, both X and Y are —C(O)NR¹—SO₂—R_{f}¹-W. In some of these embodiments, W is —SO₂Z, —CF═CF₂, —O—CF═CF₂, or —O—CF₂—CF═CF₂. In some embodiments, W is —O—CF═CF₂ or —O—CF₂—CF═CF₂. In some embodiments, at least one of X or Y is —C(O)NR¹—SO₂—R_{f}¹—SO₂Hal. In some embodiments, both X and Y are —C(O)NR¹—SO₂—R_{f}¹—SO₂Hal. In any of these embodiments, Hal is —Cl or —F.

In formula I, R is a bromine, chlorine, fluorine, or hydrogen atom. In some embodiments, R is a fluorine atom or hydrogen atom. In some embodiments, R is a fluorine atom.

In formula I, RF is a fluorinated alkenyl group that is uninterrupted or interrupted by at least one —O— group and unsubstituted or substituted by at least one chlorine atom, aryl group, or a combination thereof or RF is a fluorinated alkyl group or arylalkylenyl group that is substituted by bromine or iodine and uninterrupted or interrupted by at least one —O— group. In some embodiments, each fluorinated alkenyl group independently has up to 20, 10, 8, 6, or 4 carbon atoms. Fluorinated alkenyl groups have at least two carbon atoms, in some embodiments, at least 3 carbon atoms. Each fluorinated alkenyl may independently be partially fluorinated or perfluorinated, may have more than one alkene group, and may have up to 6, 5, 4, 3, or 2 —O— groups. In some embodiments, RF is perfluorinated alkenyl. Partially fluorinated alkenyl groups can include at least one of carbon-hydrogen bonds or carbon-chlorine bonds, for example. In some embodiments, each fluorinated alkenyl is interrupted by one —O— group. In some embodiments, the fluorinated alkenyl is not interrupted by —O— groups, and includes only carbon-carbon bonds, carbon-fluorine bonds, and optionally carbon-hydrogen or carbon-chlorine bonds. In some embodiments, RF is —CF═CF₂, —CF═CFCF₃, —CCl═CF₂, —CF═CFCl, —CF═CH₂, or —CF₂—CF═CF₂.

In some embodiments, RF is fluorinated alkenyl group that is uninterrupted or interrupted by one or more —O— groups. In some embodiments, RF is —CF═CF(CF₂)ₘ(OCₙF₂ₙ)_{z}OR_{f}², or —CF₂CF═CF(OCₙF₂n)_{z}OR_{f}³, wherein R_{f}² is a linear or branched perfluoroalkyl group having from 1 to 8 carbon atoms and uninterrupted or interrupted by one or more —O— groups; z is 0, 1, or 2; each n is independently 1, 2, 3, or 4; m is 0 or 1. In some embodiments, z is 1 or 2. In some embodiments, n is from 1 to 3, or from 2 to 3, or from 2 to 4. In some embodiments, n is 1 or 3. In some embodiments, n is 3. CₙF₂ₙ may be linear or branched. In some embodiments, CₙF₂ₙ can be written as (CF₂)ₙ, which refers to a linear perfluoroalkylene group. In some embodiments, CₙF₂ₙ is —CF₂—CF₂—CF₂—. In some embodiments, CₙF₂ₙ is branched, for example, —CF₂—CF(CF₃)—. In some embodiments, (OCₙF₂ₙ)_{z} is represented by —O—(CF₂)₁₋₄—[O(CF₂)₁₋₄]₀₋₁. In some embodiments, Rf is a linear or branched perfluoroalkyl group having from 1 to 6 carbon atoms that is optionally interrupted by up to 4, 3, or 2 —O— groups. In some embodiments, Rf is a perfluoroalkyl group having from 1 to 4 carbon atoms optionally interrupted by one —O— group.

In some embodiments, RF is a fluorinated alkyl group or arylalkylenyl group that is substituted by bromine or iodine and uninterrupted or interrupted by at least one —O— group. In some embodiments, RF is a fluorinated alkyl group that is substituted by bromine or iodine and uninterrupted or interrupted by at least one —O— group. In some embodiments, each fluorinated alkyl group independently has up to 20, 10, 8, 6, or 4 carbon atoms. In some embodiments, each fluorinated alkyl group has at least two carbon atoms, in some embodiments, at least 3 carbon atoms. Each fluorinated alkyl may independently be partially fluorinated or perfluorinated and may have up to 6, 5, 4, 3, or 2 —O— groups. In some embodiments, RF is perfluorinated alkyl substituted by at least one bromine or iodine. Partially fluorinated alkyl groups can include at least one of carbon-hydrogen bonds or carbon-chlorine bonds, for example. In some embodiments, each fluorinated alkyl is interrupted by one —O— group. In some embodiments, the fluorinated alkyl is not interrupted by —O— groups, and includes only carbon-carbon bonds, carbon-fluorine bonds, and optionally carbon-hydrogen or carbon-chlorine bonds. In these embodiments, RF can be substituted by one or two bromine atoms or one or two iodine atoms.

In some embodiments, R is a fluorine atom, and RF is a perfluorinated alkenyl group. In some embodiments, R is a fluorine atom, and RF is —CF═CF₂, —CF═CFCF₃, or —CF₂—CF═CF₂.

The process making the multifunctional compound of the present disclosure includes combining first components comprising a malonate represented by formula M′O(O)C—C(R)H—C(O)OM′, a base, and a fluorinated compound comprising an alkene group; and forming a compound represented by formula M′O(O)C—C(R)RF—C(O)OM′ (X), wherein R and RF are as defined above in any of their embodiments, and each M′ is independently alkyl or trimethylsilyl. In some embodiments, each M′ is independently alkyl having from one four carbon atoms (e.g., methyl, ethyl, n-propyl, isopropyl, n-butyl, sec-butyl, tert-butyl, or isobutyl). In some embodiments, each M′ is methyl, ethyl, or trimethylsilyl. In some embodiments, each M′ is the same. Several malonates represented by formula M′O(O)C—C(R)H—C(O)OM′ are commercially available. For example, dimethyl-2-fluoromalonate, diethyl-2-fluoromalonate, dimethyl-2-chloromalonate, diethyl-2-chloromalonate, dimethyl-2-bromomalonate, diethyl-2-bromomalonate, diethylmalonate, dimethylmalonate, and bis(trimethylsilyl)malonate are commercially available from chemical suppliers such as abcr Chemicals, Karlsruhe, Germany, and Sigma-Aldrich, St. Louis, Mo.

The first components in the method of making the multifunctional compound of the present disclosure also include a base. A variety of bases are useful in the method of the present disclosure. In some embodiments, the base comprises at least one of sodium hydride, sodium bicarbonate, potassium tert-butoxide, cesium carbonate, or n-butyl lithium. Useful bases also include ammonium and alkali-metal hydroxides. In some embodiments, the base is sodium hydride. Sodium hydride is typically available as a suspension in mineral oil and may be washed with solvent to remove mineral oil before the reaction, if desired.

In some embodiments, the fluorinated compound comprising the alkene group is R^{a}CF═CR^{a}₂, CF₂═CF—CF₂-LG, or CF₂═CF(CF₂)ₘ(OCₙF₂n)_{z}OR_{f}², wherein R^{a}, LG, R_{f}², z, n, and m are as defined below. In formula R^{a}CF═CR^{a}₂, each R^{a} is independently fluoro, chloro, bromo, hydrogen, a fluoroalkyl group (e.g. perfluoroalkyl having from 1 to 8, 1 to 4, or 1 to 3 carbon atoms and optionally interrupted by one or more oxygen atoms), fluoroalkoxy group (e.g. perfluoroalkoxy having from 1 to 8, 1 to 4, or 1 to 3 carbon atoms and optionally interrupted by one or more oxygen atoms), alkyl having up to 10 carbon atoms, alkoxy having up to 8 carbon atoms, or aryl having up to 8 carbon atoms. Examples of useful fluorinated monomers represented by formula R^{a}CF═CR^{a}₂ include vinylidene fluoride (VDF), tetrafluoroethylene (TFE), hexafluoropropylene (HFP), chlorotrifluoroethylene, 2-chloropentafluoropropene, trifluoroethylene, vinyl fluoride, dichlorodifluoroethylene, 1,1-dichlorofluoroethylene, 1-hydropentafluoropropylene, 2-hydropentafluoropropylene, tetrafluoropropylene, perfluoroalkyl perfluorovinyl ethers, perfluoroalkyl perfluoroallyl ethers, and mixtures thereof.

When RF is represented by formula —CF═CF(CF₂)ₘ(OCₙF₂ₙ)_{z}OR_{f}² or —CF₂CF═CF(OCₙF₂ₙ)_{z}OR_{f}², the fluorinated compound comprising the alkene group is represented by formula CF₂═CF(CF₂)ₘ(OCₙF₂ₙ)_{z}OR_{f}², in which m, n, z, and Rf₂ are as defined above. Suitable fluorinated compounds comprising the alkene group include those in which m and z are 0, and the perfluoroalkyl perfluorovinyl ethers are represented by formula CF₂═CFOR_{f}², wherein R is perfluoroalkyl having from 1 to 8, 1 to 4, or 1 to 3 carbon atoms, optionally interrupted by one or more —O— groups. Perfluoroalkoxyalkyl vinyl ethers suitable as fluorinated compounds comprising the alkene group include those in which m is 0 and which are represented by formula CF₂═CF(OCₙF₂ₙ)_{z}OR₄, in which each n is independently from 1 to 4, z is 1 or 2, and R_{f}² is a linear or branched perfluoroalkyl group having from 1 to 8 carbon atoms and optionally interrupted by one or more —O— groups. In some embodiments, n is from 1 to 3, or from 2 to 3, or from 2 to 4. In some embodiments, n is 1 or 3. In some embodiments, n is 3. CₙF₂ₙ may be linear or branched. In some embodiments, CₙF₂ₙ can be written as (CF₂)ₙ, which refers to a linear perfluoroalkylene group. In some embodiments, C₁F₂ₙ is —CF₂—CF₂—CF₂—. In some embodiments, CₙF₂ₙ is branched, for example, —CF₂—CF(CF₃)—. In some embodiments, (OCₙF₂ₙ)_{z} is represented by —O—(CF₂)₁₋₄—[O(CF₂)₁₋₄]₀₋₁. In some embodiments, Rf₂ is a linear or branched perfluoroalkyl group having from 1 to 8 (or 1 to 6) carbon atoms that is optionally interrupted by up to 4, 3, or 2 —O— groups. In some embodiments, Rf₂ is a perfluoroalkyl group having from 1 to 4 carbon atoms optionally interrupted by one —O— group. Suitable alkenes represented by formula CF₂═CF(OCₙF₂n)_{z}OR_{f} include perfluoromethyl vinyl ether, perfluoroethyl vinyl ether, perfluoropropyl vinyl ether, CF₂═CFOCF₂OCF₃, CF₂═CFOCF₂OCF₂CF₃, CF₂═CFOCF₂CF₂OCF₃, CF₂═CFOCF₂CF₂CF₂OCF₃, CF₂═CFOCF₂CF₂CF₂CF₂OCF₃, CF₂═CFOCF₂CF₂OCF₂CF₃, CF₂═CFOCF₂CF₂CF₂OCF₂CF₃, CF₂═CFOCF₂CF₂CF₂CF₂OCF₂CF₃, CF₂═CFOCF₂CF₂OCF₂OCF₃, CF₂═CFOCF₂CF₂OCF₂CF₂OCF₃, CF₂═CFOCF₂CF₂OCF₂CF₂CF₂OCF₃, CF₂═CFOCF₂CF₂OCF₂CF₂CF₂CF₂OCF₃, CF₂═CFOCF₂CF₂OCF₂CF₂CF₂CF₂CF₂OCF₃, CF₂═CFOCF₂CF₂(OCF₂)₃OCF₃, CF₂═CFOCF₂CF₂(OCF₂)₄OCF₃, CF₂═CFOCF₂CF₂OCF₂OCF₂OCF₃, CF₂═CFOCF₂CF₂OCF₂CF₂CF₃ CF₂═CFOCF₂CF₂OCF₂CF₂OCF₂CF₂CF₃, CF₂═CFOCF₂CF(CF₃)—O—C₃F₇(PPVE-2), CF₂═CF(OCF₂CF(CF₃))₂—O—C₃F₇(PPVE-3), and CF₂═CF(OCF₂CF(CF₃))₃—O—C₃F₇(PPVE-4). Many of these perfluoroalkoxyalkyl vinyl ethers can be prepared according to the methods described in U.S. Pat. No. 6,255,536 (Worm et al.) and U.S. Pat. No. 6,294,627 (Worm et al.).

Perfluoroalkyl alkene ethers and perfluoroalkoxyalkyl alkene ethers may also be useful as the fluorinated compound comprising the alkene group when practicing the method of the present disclosure. Suitable fluorinated olefins include those described in U.S. Pat. No. 5,891,965 (Worm et al.) and U.S. Pat. No. 6,255,535 (Schulz et al.). Such monomers include those in which n is 0 and which are represented by formula CF₂═CF(CF₂)ₘ—O—R_{f}², wherein m is 1, and wherein R_{f} is as defined above in any of its embodiments. Suitable perfluoroalkoxyalkyl allyl ethers include those represented by formula CF₂═CFCF₂(OCₙF₂ₙ)_{z}ORf₂, in which n, z, and Rf₂ are as defined above in any of the embodiments of perfluoroalkoxyalkyl vinyl ethers. Examples of suitable perfluoroalkoxyalkyl allyl ethers include CF₂═CFCF₂OCF₂CF₂OCF₃, CF₂═CFCF₂OCF₂CF₂CF₂OCF₃, CF₂═CFCF₂OCF₂OCF₃, CF₂═CFCF₂OCF₂OCF₂CF₃, CF₂═CFCF₂OCF₂CF₂CF₂CF₂OCF₃, CF₂═CFCF₂OCF₂CF₂OCF₂CF₃, CF₂═CFCF₂OCF₂CF₂CF₂OCF₂CF₃, CF₂═CFCF₂OCF₂CF₂CF₂CF₂OCF₂CF₃, CF₂═CFCF₂OCF₂CF₂OCF₂OCF₃, CF₂═CFCF₂OCF₂CF₂OCF₂CF₂OCF₃, CF₂═CFCF₂OCF₂CF₂OCF₂CF₂CF₂OCF₃, CF₂═CFCF₂OCF₂CF₂OCF₂CF₂CF₂CF₂OCF₃, CF₂═CFCF₂OCF₂CF₂OCF₂CF₂CF₂CF₂CF₂OCF₃, CF₂═CFCF₂OCF₂CF₂(OCF₂)₃OCF₃, CF₂═CFCF₂OCF₂CF₂(OCF₂)₄OCF₃, CF₂═CFCF₂OCF₂CF₂OCF₂OCF₂OCF₃, CF₂═CFCF₂OCF₂CF₂OCF₂CF₂CF₃, CF₂═CFCF₂OCF₂CF₂OCF₂CF₂OCF₂CF₂CF₃, CF₂═CFCF₂OCF₂CF(CF₃)—O—C₃F₇, and CF₂═CFCF₂(OCF₂CF(CF₃))₂—O—C₃F₇. Many of these perfluoroalkoxyalkyl allyl ethers can be prepared, for example, according to the methods described in U.S. Pat. No. 4,349,650 (Krespan).

In some embodiments, the fluorinated compound comprising the alkene group is represented by formula CF₂═CF—CF₂-LG, wherein LG is Cl, Br, I, chlorosulfate, fluorosulfate, or trifluoromethyl sulfate. Compounds represented by formula CF₂═CF—CF₂-LG can be prepared by known methods. Perfluoroallyl fluorosulfonate (CF₂═CF—CF₂—OSO₂F) can be obtained by the reaction of hexafluoropropylene with SO₃ and BF₃. CF₂═CF—CF₂—OSO₂Cl can conveniently be prepared by reaction of boron trichloride (BCl₃) and ClSO₃H to provide B(OSO₂Cl)₃ and subsequently reacting the B(OSO₂Cl)₃ and hexafluoropropylene (HFP) as described in Int. Pat. Appl. Pub. No. WO 2018/211457 (Hintzer et al.). Combining components comprising M(OSO₂CF₃)₃ and hexafluoropropylene (HFP) provides CF₂═CF—CF₂—OSO₂CF₃, wherein M is Al or B. Al(OSO₂CF₃)₃ is commercially available, for example, from chemical suppliers such as abcr GmbH (Karlsruhe, Germany) and Sigma-Aldrich (St. Louis, Mo.). Reaction of BCl₃ and CF₃SO₃H can be useful to provide B(OSO₂CF₃)₃. Further details about the preparation of CF₂═CF—CF₂—OSO₂CF₃ can be found in Int. Pat. Appl. Pub. No. WO 2018/211457 (Hintzer et al.)

Conveniently, reaction between the first components can be carried out at ambient pressure and sub-ambient temperature. In some embodiments, the first components are combined at a temperature in a range from −25° C. to 50° C. or in a range from −15° C. to 10° C. In some embodiments, the first components are allowed to react at a temperature of up to 25° C., up to 20° C., or up to 15° C. Useful reaction times include at least 30 minutes and up to 12 hours, up to 8 hours, up to 4 hours, or up to 2 hours. The reaction is typically carried out in suitable solvent. Examples of suitable solvents include polar aprotic solvents such as N,N-dimethylformamide (DMF), acetonitrile, tetrahydrofuran, dimethylsulfoxide (DMSO), N,N-dimethylacetamide (DMAC), gamma-butyrolactone, 1,2-dimethoxyethane (glyme), 1-(2-methoxyethoxy)-2-methoxyethane (diglyme), 2,5,8,11-tetraoxadodecane (triglyme), tetraglyme, dioxane, sulfolane, nitrobenzene, and benzonitrile. Combinations of any of these solvents may also be useful. In some embodiments of the process of the present disclosure, the reaction is carried out in at least one of DMF or acetonitrile. Suitable solvents can have a boiling point in a range from about 25° C. to 200° C.

In some embodiments, the process of the present disclosure further includes converting the compound represented by formula M′O(O)C—C(R)RF—C(O)OM′ to a compound represented by formula HO(O)C—C(R)RF—C(O)OH or HAL(O)C—C(R)RF—C(O)HAL. Conversion of the diester of formula M′O(O)C—C(R)RF—C(O)OM′ to a dicarboxylic acid of formula HO(O)C—C(R)RF—C(O)OH can be carried out, for example, by hydrolysis. The reaction can be carried out, for example, using a mineral acid in the presence of excess water. Suitable acids include sulfuric acid and hydrochloric acid. The reaction may be carried out at room temperature or below room temperature. Base-promoted hydrolysis of the diester of formula M′O(O)C—C(R)RF—C(O)OM′ using conventional methods may also be useful.

The dicarboxylic acid represented by formula HO(O)C—C(R)RF—C(O)OH, in which R and RF are as defined above in any of their embodiments, can be converted to the trimethylsilyl ester represented by formula (CH₃)₃SiO(O)C—C(R)RF—C(O)OSi(CH₃)₃ using conventional methods. For example, the dicarboxylic acid represented by formula HO(O)C—C(R)RF—C(O)OH can be treated with trimethylsilyl chloride in the presence of base (e.g., a tertiary amine) in a suitable solvent. The reaction can be carried out at room temperature or at an elevated temperature.

In some embodiments, the method of the present disclosure further includes converting the dicarboxylic acid of formula HO(O)C—C(R)RF—C(O)OH to a carboxylic acid halide of formula HAL(O)C—C(R)RF—C(O)HAL. In some embodiments, the dicarboxylic acid halide is a dicarboxylic acid chloride or a dicarboxylic acid fluoride. The dicarboxylic acid of formula HO(O)C—C(R)RF—C(O)OH can be converted in a dicarboxylic acid chloride of formula Cl(O)C—C(R)RF—C(O)Cl using conventional methods (e.g. reacting with phosphoryl chloride, phosphorous pentachloride, oxalylchloride, thionylchloride, or benzotrichloride). The conversion can be conveniently carried out by combining the dicarboxylic acid of formula HO(O)C—C(R)RF—C(O)OH with phosphoryl chloride and phosphorous pentachloride. The components can be combined at a sub-ambient temperature, and then the reaction can be heated at elevated temperature (e.g., at least 600 C, 70° C., 80° C., 90° C., or 100° C. or higher). The reaction can be carried out in the presence of a suitable solvent such as a polar aprotic solvent including any of those described above, and the product can be isolated by conventional methods (e.g., distillation). Similarly, the dicarboxylic acid of formula HO(O)C—C(R)RF—C(O)OH can be converted to a dicarboxylic acid fluoride of formula F(O)C—C(R)RF—C(O)F using conventional methods. The conversion can be conveniently carried out by combining the dicarboxylic acid with benzotrifluoride in the presence of catalytic iron (III) chloride. The reaction can be carried out at elevated temperature, either neat or in the presence of a suitable solvent, and the product can be isolated by conventional methods (e.g., distillation).

In some embodiments, the method of the present disclosure includes combining second components comprising the compound represented by formula F(O)C—C(R)RF—C(O)F, fluoride ion, and hexafluoropropylene oxide to provide a compound represented by formula

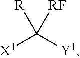

wherein X¹ is —CF₂—O—CF═CF₂, Y¹ is —C(O)F or —CF₂—O—CF═CF₂, and R and RF are as defined above in any of their embodiments. Dicarboxylic acid fluorides represented by formula F(O)C—C(R)RF—C(O)F can be converted to polyfluorinated divinyl ethers, for example, by reaction with hexafluoropropylene oxide (HFPO) in the presence of fluoride ion. The reaction can be conveniently carried out by combining the dicarboxylic acid fluoride of formula F(O)C—C(R)RF—C(O)F, HFPO, and fluoride ion at a temperature in the range of −40° C. to 60° C. depending on the catalyst used, in non-reactive organic solvents such as any of the polar aprotic solvents describe above. The molar ratio of the compound of formula F(O)C—C(R)RF—C(O)F to HFPO is in the range of 1:1 to 1:10, in some embodiments, in the range of 1:2 to 1:5. The fluoride ion can be provided by a fluoride salt. In some embodiments, the source of the fluoride ion is at least one of sodium fluoride, potassium fluoride, rubidium fluoride, cesium fluoride, or (R′)₄NF, wherein is each R¹ is independently alkyl having from 1 to 6 carbon atoms, in some embodiments, 1 to 4 or 2 to 4 carbon atoms. An embodiment of a dicarboxylic acid fluoride XV, prepared as described above, being converted into a divinyl ether XXV is shown in Reaction Scheme I, below, wherein R and RF are as defined above in any of their embodiments. In some embodiments, R is a fluorine atom, and RF is —CF═CF₂, —CF═CFCF₃, or —CF₂—CF═CF₂. When less than two equivalents of HFPO are used, X¹ is —CF₂—O—CF═CF₂, and Y¹ is —C(O)F.

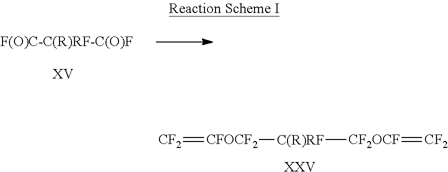

In some embodiments, the method of the present disclosure includes combining second components comprising the compound represented by formula F(O)C—C(R)RF—C(O)F, fluoride ion, and CF₂═CF—CF₂-LG, wherein LG is Cl, Br, I, chlorosulfate, fluorosulfate, or trifluoromethyl sulfate to provide a compound represented by formula

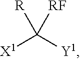

wherein X¹ is —CF₂—O—CF₂CF═CF₂, Y¹ is —C(O)F or —CF₂—O—CF₂CF═CF₂, and R and RF are as defined above in any of their embodiments. Various compounds represented by formula CF₂═CF—CF₂-LG can be prepared using the methods described above. An embodiment of a dicarboxylic acid fluoride XV, prepared as described above, being converted into a diallyl ether XXX is shown in Reaction Scheme II, below, wherein R and RF are as defined above in any of their embodiments. In some embodiments, R is a fluorine atom, and RF is —CF═CF₂, —CF═CFCF₃, or —CF₂—CF═CF₂. Divinyl ethers and diallyl ethers such as those shown in Reaction Schemes I and II can be useful, for example, for introducing long-chain branching during the preparation of fluorinated polymers as described in U.S. Pat. Appl. Pub. No. 2010/0311906 (Lavallée et al.) and/or can introduce a cure site into a fluoropolymer for crosslinking.

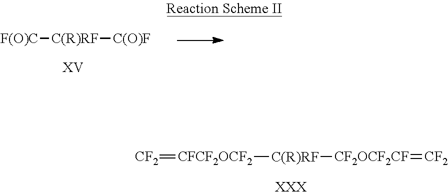

Compounds represented by XXX can be made, for example, by reacting dicarboxylic acid fluoride represented by formula XV with perfluoroallyl chloride, perfluoroallyl bromide, perfluoroallyl iodide, or perfluoroallyl fluorosulfate in the presence of potassium fluoride as described in U.S. Pat. No. 4,273,729 (Krespan). Compounds represented by formula XXX can also be prepared by combining dicarboxylic acid fluoride represented by formula XV, at least one of CF₂═CF—CF₂—OSO₂Cl or CF₂═CF—CF₂—OSO₂CF₃, and fluoride ion. The fluoride ion can be provided by a fluoride salt. In some embodiments, the source of the fluoride ion is at least one of sodium fluoride, potassium fluoride, rubidium fluoride, cesium fluoride, or (R′)₄NF, wherein is each R¹ is independently alkyl having from 1 to 6 carbon atoms, in some embodiments, 1 to 4 or 2 to 4 carbon atoms. Suitable solvents for the transformation include polar, aprotic solvents, for example, any of those described above. When less than two equivalents of CF₂═CF—CF₂-LG are used, X¹ is —CF₂—O—CF—CF═CF₂, and Y¹ is —C(O)F.

For the reaction of dicarboxylic acid fluoride such as XV, shown in Reaction Schemes I and II, one or two equivalents of components other than the dicarboxylic acid fluoride may be useful for converting the dicarboxylic acid fluoride into a multifunctional fluorinated compound in which the functional groups are different or the same, respectively. The desired number of equivalents of components other than the polyfluorinated dicarboxylic acid fluoride may be exceeded in some cases by up to 10 mol %, 7.5 mol %, or 5 mol %.

One or more of the fluorinated divinyl ethers in a compound of formula XXV and allyl ethers of formula XXX can be reacted with a mixture of elemental iodine and iodine pentafluoride at a temperature in the range of 30° C. to 200° C., or in the range of 80° C. to 160° C. Alternatively, the reaction of the resulting divinyl ether could be conducted with ICl, HF, and BF₃ at about 50° C. to form further multifunctional fluorinated compounds. Examples of such compounds include I—CF₂—CF₂—O—CF₂—C(R)RF—CF₂—O—CF═CF₂, I—CF₂—CF₂—O—CF₂—C(R)RF—CF₂—O—CF₂—CF₂—1, I—CF₂—CF₂—CF₂—O—CF₂—C(R)RF—CF₂—O—CF₂CF═CF₂, and I—CF₂—CF₂—CF₂—O— CF₂—C(R)RF—CF₂—O—CF₂—CF₂—CF₂—I. The alkene group in RF may also be iodinized under this reaction conditions to provide compounds in which RF is a fluorinated alkyl group or arylalkylenyl group that is substituted by iodine and uninterrupted or interrupted by at least one —O— group. These compounds can also be useful for crosslinking fluoropolymers.

Dicarboxylic acid fluorides and compounds represented by formula M′O(O)C—C(R)RF—C(O)OM′ (X) can also be converted into nitriles as shown in Reaction Scheme III below, where R and RF are as defined above in any of their embodiments. In some embodiments, R is a fluorine atom, and RF is —CF═CF₂, —CF═CFCF₃, or —CF₂—CF═CF₂. Amination and subsequent oxidation (e.g. with P₄O₁₀) as described, for example, in EP0710645A1 (1996) and EP0708139A1 (1996) can be useful for converting the compounds of formula M′O(O)C—C(R)RF—C(O)OM′ into the nitrile XXXV. In other embodiments, one of the acid fluoride groups in a compound of formula XVI can be converted to a polyfluorinated vinyl ether or allyl ether, for example, using the processes described above in connection with Reaction Schemes I and II, and the other of the acid fluoride groups can be converted into a cyano group by known methods [e.g., esterification (e.g. with CH₃OH), amination (e.g., with ammonia) and subsequent oxidation (e.g. with P₄O₁₀) as described, for example, in EP0710645A1 (1996) and EP0708139A1 (1996). Cure site monomers such as XXXV shown in Reaction Scheme III can be useful, for example, for introducing a cure site into a fluoropolymer for crosslinking.

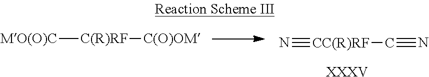

In some embodiments of the multifunctional compound of the present disclosure and/or made by the process of the present disclosure, at least one of X or Y (or X² or Y²) is —C(O)—NR¹SO₂R_{f}¹SO₂Z, wherein R¹, R_{f}¹, and Z are as defined above in any of their embodiments. These compounds can be made, for example, starting with a diester represented by formula M′O(O)C—C(R)RF—C(O)OM′ or a dicarboxylic acid fluoride of formula XV, wherein R and RF are as defined above in any of their embodiments. In some embodiments, R is a fluorine atom, and RF is —CF═CF₂, —CF═CFCF₃, or —CF₂—CF═CF₂. The diester or dicarboxylic acid fluorinated can be aminated with ammonia or a primary or secondary amine represented by formula R¹₂N, wherein each R¹ is independently hydrogen or alkyl having up to 4 carbon atoms. The reaction can conveniently be carried out at ambient temperature or below ambient temperature, optionally in any of the polar aprotic solvents described above. Before or after reaction with the amine represented by formula R¹₂N, the alkene group in RF can be protected, if desired, by reacting the alkene with bromine or chlorine to form a dibromo- or dichloro-compound using methods described, for example, in EP0710645A1 (1996).

The resulting amides, represented by formula XVI, can be further reacted with multifunctional sulfonyl fluoride or sulfonyl chloride compounds XVIII to provide the fluorinated imides XL shown in Reaction Scheme IV. When amide XVI is a tertiary amide with both R¹ groups being alkyl, the amide groups in formula XL will have quaternary ammonium groups. Typically, at least R₁ group is hydrogen. Examples of useful multi-functional compounds represented by formula XVIII include 1,1,2,2-tetrafluoroethyl-1,3-disulfonyl fluoride; 1,1,2,2,3,3-hexafluoropropyl-1,3-disulfonyl fluoride; 1,1,2,2,3,3,4,4-octafluorobutyl-1,4-disulfonyl fluoride; 1,1,2,2,3,3,4,4,5,5-perfluorobutyl-1,5-disulfonyl fluoride; 1,1,2,2-tetrafluoroethyl-1,2-disulfonyl chloride; 1,1,2,2,3,3-hexafluoropropyl-1,3-disulfonyl chloride; 1,1,2,2,3,3,4,4-octafluorobutyl-1,4-disulfonyl chloride; and 1,1,2,2,3,3,4,4,5,5-perfluorobutyl-1,5-disulfonyl chloride. Sulfonyl halide groups can be hydrolyzed or treated with further compound represented by formula R¹₂N to provide compounds of formula XL, wherein Z is as defined above. Hydrolysis of a copolymer having —SO₂F groups with an alkaline hydroxide (e.g. LiOH, NaOH, or KOH) solution provides —SO₃Z groups, which may be subsequently acidified to SO₃H groups. Treatment of a compound having —SO₂F groups with water and steam can form SO₃H groups.

If the alkene in the RF group is protected with bromine before carrying out Reaction Scheme IV, deprotection can be carried out, if desired, with zinc powder, for example, in a suitable solvent (e.g., any of the polar aprotic solvents described above). Further details regarding the deprotection can be found, for example, in in EP0710645A1 (1996). The deprotection can be carried out at elevated temperature, and the product of formula XL can be isolated using convention methods. If deprotection is not carried out RF can be a fluorinated alkyl group or arylalkylenyl group that is substituted by bromine and uninterrupted or interrupted by at least one —O— group.

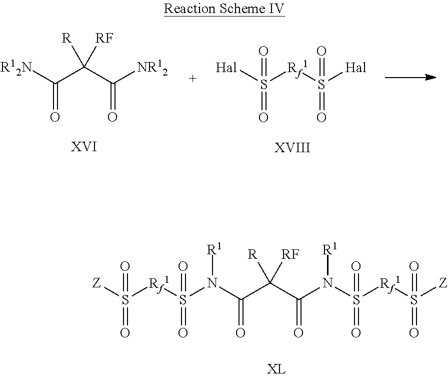

Compounds of Formula XVI can also be treated with polysulfonimides represented by formula FSO₂(CF₂)ₐ[SO₂NZSO₂(CF₂)ₐ]₁₋₁₀S®₂F or FSO₂(CF₂)ₐ[SO₂NZSO₂(CF₂)ₐ]₁₋₁₀S®₃H, wherein each a is independently 1 to 6, 1 to 4, or 2 to 4. To make a polysulfonimide, a sulfonyl halide monomer (e.g., any of those described above) and a sulfonamide monomer represented by formula H₂NSO₂(CF₂)ₐSO₂NH₂ are made to react in the mole ratio of (k+1)/k. The reaction may be carried out, for example, in a suitable solvent (e.g., acetonitrile) at 0° C. in the presence of base. The sulfonyl halide monomer and sulfonamide monomer may have the same or different values of a, resulting in the same or different value of a for each repeating unit. The resulting product FSO₂(CF₂)ₐ[SO₂NZSO₂(CF₂)ₐ]₁₋₁₀SO₂F may be treated with one equivalent of water in the presence of base (e.g., N,N-diisopropylethylamine (DIPEA)) to provide FSO₂(CF₂)ₐ[SO₂NZSO₂(CF₂)ₐ]₁₋₁₀S®₃H, as described in JP 2011-40363.

Amides represented by formula XVI can also be reacted with other sulfonyl fluoride or sulfonyl chloride compounds XIX to provide the fluorinated imides XLV shown in Reaction Scheme V, below, wherein R, RF, R¹, R_{f}¹, and W are as defined above.

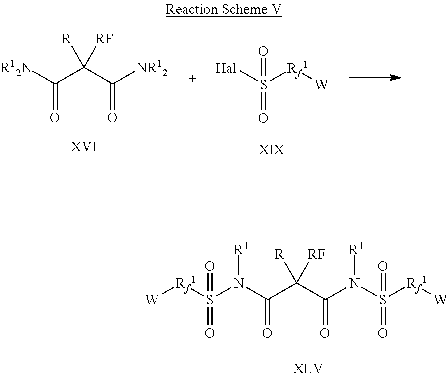

Some compounds of formula XIX, suitable for reaction with amides using the process of Reaction Scheme V can be represented by formula CF₂═CF—(CF₂)₀₋₁—SO₂Hal or CF₂═CF(CF₂)₀₋₁(OC_{b}F_{2b})_{c}—O—(CₑF₂ₑ)—SO₂Hal, wherein Hal is —Cl or —F. In formula CF₂═CFCF₂—(OC_{b}F_{2b})_{c}—O—(CₑF₂ₑ)—SO₂Hal, b is a number from 2 to 8, 0 or 2, and e is a number from 1 to 8. In some embodiments, b is a number from 2 to 6 or 2 to 4. In some embodiments, b is 2. In some embodiments, e is a number from 1 to 6 or 2 to 4. In some embodiments, e is 2. In some embodiments, e is 4. In some embodiments, c is 0 or 1. In some embodiments, c is 0. In some embodiments, c is 0, and e is 2 or 4. In some embodiments, b is 3, c is 1, and e is 2. CₑF₂ₑ may be linear or branched. In some embodiments, CₑF₂ₑ can be written as (CF₂)ₑ, which refers to a linear perfluoroalkylene group. When c is 2, the b in the two C_{b}F_{2b} groups may be independently selected. However, within a C_{b}F_{2b} group, a person skilled in the art would understand that b is not independently selected. Examples of useful compounds represented by formula CF₂═CFCF₂—(OC_{b}F_{2b})_{c}—O—(CₑF₂ₑ)—SO₂Z include CF₂═CFCF₂—O—CF₂—SO₂Z, CF₂═CFCF₂—O—CF₂CF₂—SO₂Z, CF₂═CFCF₂—O—CF₂CF₂CF₂—SO₂Z, CF₂═CFCF₂—O—CF₂CF₂CF₂CF₂—SO₂Z, and CF₂═CFCF₂—O—CF(CF₃)—CF₂—O—(CF₂)ₑ—SO₂Z.

Compounds represented by formula CF₂═CF(CF₂)ₐ—(OC_{b}F_{2b})_{c}—O—(CₑF₂ₑ)—SO₂Z can be made by known methods. For example acid fluorides represented by formula FSO₂(CF₂)ₑ₋₁—C(O)F or FSO₂(CF₂)ₑ—(OC_{b}F_{2b})_{c-1}—C(O)F can be reacted with perfluoroallyl chloride, perfluoroallyl bromide, or perfluoroallyl fluorosulfate in the presence of potassium fluoride as described in U.S. Pat. No. 4,273,729 (Krespan) to make compounds of formula CF₂═CFCF₂—(OC_{b}F_{2b})_{c}—O—(CₑF₂ₑ)—SO₂F. Compounds of formula CF₂═CFCF₂—(OC_{b}F_{2b})_{c}—O—(CₑF₂ₑ)—SO₂F can be hydrolyzed with a base (e.g., alkali metal hydroxide or ammonium hydroxide) to provide a compound represented by formula CF₂═CFCF₂—(OC_{b}F_{2b})_{c}—O—(CₑF₂ₑ)—SO₃Z.

Multifunctional compounds of the present disclosure and/or made by the process of the present disclosure are useful, for example, in the preparation of fluoropolymers. For example, the multifunctional compounds can be interpolymerized with at least one partially fluorinated or perfluorinated ethylenically unsaturated monomer represented by formula R^{a}CF═CR^{a}₂, wherein each R^{a} is independently fluoro, chloro, bromo, hydrogen, a fluoroalkyl group (e.g. perfluoroalkyl having from 1 to 8, 1 to 4, or 1 to 3 carbon atoms and optionally interrupted by one or more oxygen atoms), fluoroalkoxy group (e.g. perfluoroalkoxy having from 1 to 8, 1 to 4, or 1 to 3 carbon atoms and optionally interrupted by one or more oxygen atoms), alkyl having up to 10 carbon atoms, alkoxy having up to 8 carbon atoms, or aryl having up to 8 carbon atoms. Examples of useful fluorinated monomers represented by formula R^{a}CF═CR^{a}₂ include vinylidene fluoride (VDF), tetrafluoroethylene (TFE), hexafluoropropylene (HFP), chlorotrifluoroethylene, 2-chloropentafluoropropene, trifluoroethylene, vinyl fluoride, dichlorodifluoroethylene, 1,1-dichlorofluoroethylene, 1-hydropentafluoropropylene, 2-hydropentafluoropropylene, tetrafluoropropylene, perfluoroalkyl perfluorovinyl ethers, perfluoroalkyl perfluoroallyl ethers, and mixtures thereof.

In some embodiments, the fluoropolymer that includes units derived from a multifunctional compound of the present disclosure includes units from one or more monomers independently represented by formula CF₂═CF(CF₂)ₘ(OCₙF₂ₙ)_{z}OR_{f}², in which m, n, z, and Rf₂ are as defined above. Suitable monomers of this formula include those in which m and z are 0, and the perfluoroalkyl perfluorovinyl ethers are represented by formula CF₂═CFOR_{f}², wherein R_{f}² is perfluoroalkyl having from 1 to 8, 1 to 4, or 1 to 3 carbon atoms, optionally interrupted by one or more —O— groups. Perfluoroalkoxyalkyl vinyl ethers suitable for making a fluoropolymer include those represented by formula CF₂═CF(CF₂)ₘ(OCₙF₂ₙ)_{z}OR_{f}², in which m is 0, each n is independently from 1 to 6, z is 1 or 2, and R_{f}² is a linear or branched perfluoroalkyl group having from 1 to 8 carbon atoms and optionally interrupted by one or more —O— groups. In some embodiments, n is from 1 to 4, or from 1 to 3, or from 2 to 3, or from 2 to 4. In some embodiments, n is 1 or 3. In some embodiments, n is 3. CₙF₂ₙ may be linear or branched. In some embodiments, CₙF₂ₙ can be written as (CF₂)ₙ, which refers to a linear perfluoroalkylene group. In some embodiments, CₙF₂ₙ is —CF₂—CF₂—CF₂—. In some embodiments, CₙF₂ₙ is branched, for example, —CF₂—CF(CF₃)—. In some embodiments, (OCₙF₂n)_{z} is represented by —O—(CF₂)₁₋₄—[O(CF₂)₁₋₄]₀₋₁. In some embodiments, R_{f}² is a linear or branched perfluoroalkyl group having from 1 to 8 (or 1 to 6) carbon atoms that is optionally interrupted by up to 4, 3, or 2 —O— groups. In some embodiments, R_{f}² is a perfluoroalkyl group having from 1 to 4 carbon atoms optionally interrupted by one —O— group. Suitable monomers represented by formula CF₂═CF_{f}² and CF₂═CF(OCₙF₂n)_{z}OR_{f} include perfluoromethyl vinyl ether, perfluoroethyl vinyl ether, perfluoropropyl vinyl ether, CF₂═CFOCF₂OCF₃, CF₂═CFOCF₂OCF₂CF₃, CF₂═CFOCF₂CF₂OCF₃, CF₂═CFOCF₂CF₂CF₂OCF₃, CF₂═CFOCF₂CF₂CF₂CF₂OCF₃, CF₂═CFOCF₂CF₂OCF₂CF₃, CF₂═CFOCF₂CF₂CF₂OCF₂CF₃, CF₂═CFOCF₂CF₂CF₂CF₂OCF₂CF₃, CF₂═CFOCF₂CF₂OCF₂OCF₃, CF₂═CFOCF₂CF₂OCF₂CF₂OCF₃, CF₂═CFOCF₂CF₂OCF₂CF₂CF₂OCF₃, CF₂═CFOCF₂CF₂OCF₂CF₂CF₂CF₂OCF₃, CF₂═CFOCF₂CF₂OCF₂CF₂CF₂CF₂CF₂OCF₃, CF₂═CFOCF₂CF₂(OCF₂)₃OCF₃, CF₂═CFOCF₂CF₂(OCF₂)₄OCF₃, CF₂═CFOCF₂CF₂OCF₂OCF₂OCF₃, CF₂═CFOCF₂CF₂OCF₂CF₂CF₃ CF₂═CFOCF₂CF₂OCF₂CF₂OCF₂CF₂CF₃, CF₂═CFOCF₂CF(CF₃)—O—C₃F₇ (PPVE-2), CF₂═CF(OCF₂CF(CF₃))₂—O—C₃F₇(PPVE-3), and CF₂═CF(OCF₂CF(CF₃))₃—O—C₃F₇(PPVE-4). Many of these perfluoroalkoxyalkyl vinyl ethers can be prepared according to the methods described in U.S. Pat. No. 6,255,536 (Worm et al.) and U.S. Pat. No. 6,294,627 (Worm et al.).

Suitable fluoro (alkene ether) monomers include those described in U.S. Pat. No. 5,891,965 (Worm et al.) and U.S. Pat. No. 6,255,535 (Schulz et al.). Such monomers include those in which n is 0 and which are represented by formula CF₂═CF(CF₂)ₘ—O—R_{f}², wherein m is 1, and wherein Rf is as defined above in any of its embodiments. Suitable perfluoroalkoxyalkyl allyl ethers include those represented by formula CF₂═CFCF₂(OCₙF₂ₙ)_{z}ORf₂, in which n, z, and Rf₂ are as defined above in any of the embodiments of perfluoroalkoxyalkyl vinyl ethers. Examples of suitable perfluoroalkoxyalkyl allyl ethers include CF₂═CFCF₂OCF₂CF₂OCF₃, CF₂═CFCF₂OCF₂CF₂CF₂OCF₃, CF₂═CFCF₂OCF₂OCF₃, CF₂═CFCF₂OCF₂OCF₂CF₃, CF₂═CFCF₂OCF₂CF₂CF₂CF₂OCF₃, CF₂═CFCF₂OCF₂CF₂OCF₂CF₃, CF₂═CFCF₂OCF₂CF₂CF₂OCF₂CF₃, CF₂═CFCF₂OCF₂CF₂CF₂CF₂OCF₂CF₃, CF₂═CFCF₂OCF₂CF₂OCF₂OCF₃, CF₂═CFCF₂OCF₂CF₂OCF₂CF₂OCF₃, CF₂═CFCF₂OCF₂CF₂OCF₂CF₂CF₂OCF₃, CF₂═CFCF₂OCF₂CF₂OCF₂CF₂CF₂CF₂OCF₃, CF₂═CFCF₂OCF₂CF₂OCF₂CF₂CF₂CF₂CF₂OCF₃, CF₂═CFCF₂OCF₂CF₂(OCF₂)₃OCF₃, CF₂═CFCF₂OCF₂CF₂(OCF₂)₄OCF₃, CF₂═CFCF₂OCF₂CF₂OCF₂OCF₂OCF₃, CF₂═CFCF₂OCF₂CF₂OCF₂CF₂CF₃, CF₂═CFCF₂OCF₂CF₂OCF₂CF₂OCF₂CF₂CF₃, CF₂═CFCF₂OCF₂CF(CF₃)—O—C₃F₇, and CF₂═CFCF₂(OCF₂CF(CF₃))₂—O—C₃F₇. Many of these perfluoroalkoxyalkyl allyl ethers can be prepared, for example, according to the methods described in U.S. Pat. No. 4,349,650 (Krespan).

Perfluoro-1,3-dioxoles may also be useful to prepare a fluoropolymer that includes units derived from a multifunctional compound of the present disclosure. Perfluoro-1,3-dioxole monomers and their copolymers are described in U.S. Pat. No. 4,558,141 (Squire).

The multifunctional compounds disclosed herein can be useful for preparing amorphous fluoropolymers, semi-crystalline thermoplastics, and non-melt processable fluoroplastics.

In some embodiments, one or more multifunctional compounds disclosed herein can be copolymerized with TFE to form a non-melt processable fluoroplastic. The multifunctional compound may be any of those described above. In a non-melt processable fluoroplastic, one or more of the multifunctional compounds are included in the monomers for polymerization in an amount of up to about one percent by weight. TFE homo- and copolymers including a comonomer in an amount of up to about one percent by weight are referred to in the art as PTFE. PTFE has such a high melt viscosity and/or low melt flow index (MFI) that it cannot be processed by conventional melt processing techniques such as extrusion, injection molding, or blow molding. In some embodiments, the fluoropolymer contains TFE units and units from at least one multifunctional compound and no other comonomer units. The amount of the multifunctional compound comonomer units may be up to 1% by weight or up to 0.10% by weight. For example, the amount of the multifunctional compound comonomer units can be from 0.01 to 1 percent by weight or from 0.3 to 1 percent by weight, based on the total weight of the fluoropolymer (in which the comonomer units add up to give 100% by weight).

The molecular weights of certain fluoroplastics are often characterized by the melt viscosity or the melt flow index (MFI; e.g., 372° C./5 kg). In some embodiments, the non-melt-processable fluoropolymer made from the multifunctional polyfluorinated compound has a melt flow index (MFI) of 1.0 g/10 min or less at 372° C. using a 5 kg load (MFI 372/5 of less than 1.0 g/10 min), in some embodiments, a melt flow index (372/5) of 0.1 g/10 minutes or less. In some embodiments, the non-melt-processable fluoropolymer has a melting point of at least 300° C., in some embodiments, at least 315° C., and typically within the range of 327+/−10° C. In some embodiments, the non-melt-processable fluoropolymer has a melting point of at least 317° C., at least 319° C., or at least 321° C. The melting point of not melt-processable fluoropolymers differs when the material is molten for the first time and after subsequent melting. After the material has been molten once, the meting point in subsequent melting remains constant. The melting point as referred to herein is the melting point of previously molten material (i.e., the material was brought to the melting point, cooled below its melting point, and then melted again).

PTFEs made with one or more multifunctional compounds disclosed herein can be useful, for example, for gaskets and inner liners for pipes and containers.

In some embodiments, one or more of the multifunctional compounds can be copolymerized with TFE to form a fluorothermoplastic. Copolymers of TFE and perfluorinated vinyl or allyl ethers are known in the art as PFA's (perfluorinated alkoxy polymers). In these embodiments, the fluorinated vinyl or allyl ether units are present in the copolymer in an amount in a range from 0.5 mol % to 15 mol %, in some embodiments, 0. 5 mol % to 10 mol %, and in some embodiments, 0.5 mol % to 5 mol %. The multifunctional vinyl or allyl ethers described above in any of their embodiments, for example, compounds represented by formula

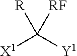

can be useful in the preparation of PFAs. In some embodiments, the copolymer of TFE and at least one fluorinated vinyl ether or allyl ether consists essentially of units derived from TFE and at least one of the multifunctional compounds disclosed herein. “Consisting essentially of” as used herein refers to the absence of other comonomers or the presence of units derived from other comonomers in an amount of less than one percent by weight, in some embodiments, less than 0.1 percent by weight. In some embodiments, the copolymer of TFE and at least one of the multifunctional compounds further comprises at least one percent by weight, in some embodiments, up to 10, 6, 5, or 4 percent by weight of other units derived from compounds represented by formula R^{a}CF═CR^{a}₂ described above, non-fluorinated olefins (e.g., ethene or propene). In some embodiments, at least one of HFP, VDF, vinyl fluoride, chlorotrifluoroethylene, ethene, or propene is included in the monomers in an amount up to ten percent by weight to make the fluorothermoplastic. In some embodiments, the fluorothermoplastic made from at least one of the multifunctional compounds disclosed herein has a melt flow index (MFI) in a range from 0.5 g/10 min to 100 g/10 min at 372° C. using a 5 kg load (MFI 372/5 of in a range from 0.5 g/10 min to 100 g/10 min). In some embodiments, the copolymer has a melting point of from 200° C. to 310° C. and a melt flow index (MFI at 372° C. and 5 kg load) of 0.5 to 19 grams/10 minutes. In some embodiments, the copolymer has a melting point of from 250° C. to 290° C. and have a melt flow index (MFI at 372° C. and 5 kg load) of from 30 grams/10 minutes to 50 grams/10 minutes.

In some embodiments, one or more of the multifunctional compounds disclosed herein can be copolymerized with TFE and HFP. The multifunctional compound may be any of those described above. Copolymers of TFE and HFP with or without other perfluorinated comonomers are known in the art as FEP's (fluorinated ethylene propylene). In some embodiments, these fluorothermoplastics are derived from copolymerizing 30 to 70 wt. % TFE, 10 to 30 wt. %, HFP, and 0.2 to 50 wt. % of other comonomers, which can include one or more of the multifunctional compounds disclosed herein. These weight percentages are based on the weight of the polymer, and the comonomers add up to give 100% by weight. In some embodiments, units derived from the multifunctional compounds disclosed herein are present in the copolymer according to the present disclosure in a range from 0.2 percent by weight to 12 percent by weight, based on the total weight of the copolymer. In some embodiments, units derived from the multifunctional compound are present in a range from 0.5 percent by weight to 6 percent by weight, based on the total weight of the copolymer, with the total weight of the copolymer being 100% by weight. In some embodiments, units derived from the multifunctional compound are present in the copolymer according to the present disclosure in a range from 0.02 mole percent to 2 mole percent, based on the total amount of the copolymer. In some embodiments, units derived from the multifunctional compound are present in the copolymer in an amount up to 1.5 mole percent or up to 1.0 mole percent. In some embodiments, the copolymerized units derived from multifunctional compound are present in the copolymer in an amount of at least 0.03 mole percent or 0.05 mole percent. The copolymerized units derived from may be present in the copolymer in a range from 0.02 mole percent to 2 mole percent, 0.03 mole percent to 1.5 mole percent, or 0.05 mole percent to 1.0 mole percent. The HFP may be present in a range from 5 wt. % to 22 wt. %, in a range from 10 wt. % to 17 wt. %, in a range from 11 wt. % to 16 wt. %, or in a range from 11.5 wt. % to 15.8 wt. %, based on the total weight of the copolymer, wherein the weight of the copolymer is 100% by weight. The copolymers made according to the methods of the present disclosure typically have a melting point between 220° C. to 285° C., in some embodiments, 235° C. to 275° C., 240° C. to 275° C., or 245° C. to 265 T. In some embodiments, the copolymer prepared from the multifunctional compound, TFE, and HFP has an MFI at 372° C. and 5 kg load of 30±10 grams per 10 minutes. In some embodiments, the copolymer prepared from the multifunctional compound, TFE, and HFP has an MFI at 372° C. and 5 kg load of 30±5 grams per 10 minutes or 30±3 grams per 10 minutes. In some embodiments, the copolymer prepared from the multifunctional compound, TFE, and HFP has an MFI at 372° C. and 5 kg load in a range from 1 gram per 10 minutes to 19 grams per 10 minutes. In some embodiments, this copolymer has an MFI in a range from 1 gram per 10 minutes to 15 grams per 10 minutes or in a range from 1 gram per 10 minutes to 10 grams per 10 minutes.

FEPs made with one or more multifunctional compounds disclosed herein can be useful, for example, for electrical insulation in Local Area Networks (LAN).

In some embodiments, one or more multifunctional compounds of the present disclosure and/or made by the process disclosed herein can be used to make amorphous fluoropolymers. Amorphous fluoropolymers typically do not exhibit a melting point and exhibit little or no crystallinity at room temperature. Useful amorphous fluoropolymers can have glass transition temperatures below room temperature or up to 280° C. Suitable amorphous fluoropolymers can have glass transition temperatures in a range from −60° C. up to 280° C., −60° C. up to 250° C., from −60° C. to 150° C., from −40° C. to 150° C., from −40° C. to 100° C., or from −40° C. to 20° C.

In some embodiments, amorphous fluoropolymers that include units derived from a multifunctional compound disclosed herein include a TFE/propylene copolymer, a TFE/propylene/VDF copolymer, a VDF/HFP copolymer, a TFE/VDF/HFP copolymer, a TFE/perfluoromethyl vinyl ether (PMVE) copolymer, a TFE/CF₂═CFOC₃F₇ copolymer, a TFE/CF₂═CFOCF₃/CF₂═CFOC₃F₇ copolymer, a TFE/ethyl vinyl ether (EVE) copolymer, a TFE/butyl vinyl ether (BVE) copolymer, a TFE/EVE/BVE copolymer, a VDF/CF₂═CFOC₃F₇ copolymer, an ethylene/HFP copolymer, a TFE/HFP copolymer, a CTFE/VDF copolymer, a TFE/VDF copolymer, a TFE/perfluoro-1,3-dioxole copolymer, a TFE/VDF/PMVE/ethylene copolymer, or a TFE/VDF/CF₂═CFO(CF₂)₃OCF₃ copolymer.

In some embodiments, the amorphous fluoropolymer that includes units derived from a multifunctional compound disclosed herein includes polymerized units comprising a cure site. In these embodiments, cure site monomers (including any of those described above, for example, compounds of formula XXV, XXX, XXXV, I—CF₂—CF₂—O—CF₂—C(R)RF—CF₂—O—CF═CF₂, I—CF₂—CF₂—O—CF₂—C(R)RF—CF₂—O—CF₂—CF₂—I, I—CF₂—CF₂—CF₂—O—CF₂—C(R)RF—CF₂—O—CF₂CF═CF₂, I—CF₂—CF₂—CF₂—O—CF₂—C(R)RF—CF₂—O—CF₂—CF₂—CF₂—I, and brominated analogues of any of these iodo compounds may be useful during the polymerization to make the amorphous fluoropolymer. In some embodiments, RF is perfluorinated, which may be useful, for example, for increasing thermal stability of the resulting elastomer. Such cure site monomers include those monomers capable of free radical polymerization. Examples of useful cure sites include a Br cure site, an I cure site, a nitrile cure site, a carbon-carbon double bond, and combinations thereof. Any of these cure sites can be cured using peroxides, for example. However, in some cases in which multiple, different cure sites are present a dual cure system or a multi cure system may be useful. Other suitable cure systems that may be useful include bisphenol curing systems or triazine curing systems. Triazine curing systems may be useful, for example, with amorphous fluoropolymers having units derived from multifunctional compounds represented by formula XXXV, for example. Useful amounts of the cure site monomers include 0.01 mol % to 1 mol %, based on total moles of monomer incorporated into the polymer may be used. In some embodiments, at least 0.02, 0.05, or even 0.1 mol % of a cure site monomer is used and at most 0.5, 0.75, or even 0.9 mol % of a cure site monomer is used based on the total moles of monomer incorporated into the amorphous fluoropolymer.

In some embodiments, the amorphous fluoropolymer that includes units derived from a multifunctional compound disclosed herein includes polymerized units comprising at least one compound represented by XXV or XXX as a cure site monomer, as described above in any of its embodiments. The compound represented by formula XXV or XXX may be present in the components to be polymerized in any useful amount, in some embodiments, in an amount of up to 2, 1, or 0.5 mole percent and in an amount of at least 0.1 mole percent, based on the total amount of polymerizable components.

If the amorphous fluoropolymer is perhalogenated, in some embodiments perfluorinated, typically at least 50 mole percent (mol %) of its interpolymerized units are derived from TFE and/or CTFE, optionally including HFP. The balance of the interpolymerized units of the amorphous fluoropolymer (e.g., 10 to 50 mol %) is made up of one or more perfluorinated vinyl or allyl ethers, and the multifunctional compound of the present disclosure as the cure site monomer. If the fluoropolymer is not perfluorinated, it typically contains from about 5 mol % to about 90 mol % of its interpolymerized units derived from TFE, CTFE, and/or HFP; from about 5 mol % to about 90 mol % of its interpolymerized units derived from VDF, ethylene, and/or propylene; up to about 40 mol % of its interpolymerized units derived from perfluorinated vinyl or allyl ethers; and from about 0.1 mol % to about 5 mol %, in some embodiments from about 0.3 mol % to about 2 mol %, of the multifunctional compound of the present disclosure as the cure site monomer.

In some embodiments of the method of making the fluoropolymer according to the present disclosure, the method includes crosslinking the fluoropolymer to make a fluoroelastomer. In these embodiments, the fluoropolymer prepared from a multifunctional compound of the present disclosure as the cure site monomer is formulated into a curable composition. In some embodiments, the curable composition includes a peroxide. Suitable peroxides are generally those which generate free radicals at curing temperatures. Dialkyl peroxides and bis(dialkyl peroxides), each of which decomposes at a temperature above 50° C., may be useful. Examples of useful peroxides include 2,5-dimethyl-2,5-di(t-butylperoxy)hexyne-3, 2,5-dimethyl-2,5-di(t-butylperoxy)hexane, dicumyl peroxide, t-butyl perbenzoate, a,a′-bis(t-butylperoxy-diisopropylbenzene), and di[1,3-dimethyl-3-(t-butylperoxy)-butyl]carbonate. Acyl peroxides tend to decompose at lower temperatures than alkyl peroxides and allow for lower temperature curing. Examples of useful acyl peroxides include di(4-t-butylcyclohexyl)peroxydicarbonate, di(2-phenoxyethyl)peroxydicarbonate, di(2,4-dichlorobenzoyl) peroxide, dilauroyl peroxide, decanoyl peroxide, 1,1,3,3-tetramethylethylbutylperoxy-2-ethylhexanoate, 2,5-dimethyl-2,5-di(2-ethylhexanoylperoxy)hexane, disuccinic acid peroxide, t-hexyl peroxy-2-ethylhexanoate, di(4-methylbenzoyl) peroxide, t-butyl peroxy-2-ethylhexanoate, benzoyl peroxide, t-butylperoxy 2-ethylhexyl carbonate, and t-butylperoxy isopropyl carbonate.

Furthermore, in peroxide-cured fluoroelastomers, it is often desirable to include a crosslinker. The crosslinkers may be useful, for example, for providing enhanced mechanical strength in the final cured composition. Examples of useful crosslinkers include tri(methyl)allyl isocyanurate (TMAIC), triallyl isocyanurate (TAIC), tri(methyl)allyl cyanurate, poly-triallyl isocyanurate (poly-TAIC), xylylene-bis(diallyl isocyanurate) (XBD), N,N′-m-phenylene bismaleimide, diallyl phthalate, tris(diallylamine)-s-triazine, triallyl phosphite, 1,2-polybutadiene, ethyleneglycol diacrylate, diethyleneglycol diacrylate, and CH₂═CH—R_{f1}—CH═CH₂, wherein R_{f1} is a perfluoroalkylene having from 1 to 8 carbon atoms. The crosslinker is typically present in an amount of 1% by weight to 10% by weight versus the weight of the fluoropolymer composition. In some embodiments, the crosslinker is present in a range from 2% by weight to 5% by weight versus the weight of the fluoropolymer composition.

Curing a curable fluoropolymer having nitrogen-containing cure sites (e.g., wherein at least one of X or Y is —C≡N) can be carried out using organo onium catalysts such as those described in U.S. Pat. No. 8,906,821 (Grootaert). Curable compositions including fluoropolymers having nitrogen-containing cure sites can also be modified by using yet other types of curatives. Examples of such curatives for amorphous fluoropolymers with nitrile cure sites include bis-aminophenols (e.g., U.S. Pat. No. 5,767,204 (Iwa et al.) and U.S. Pat. No. 5,700,879 (Yamamoto et al.)), bis-amidooximes (e.g., U.S. Pat. No. 5,621,145 (Saito et al.)), and ammonium salts (e.g., U.S. Pat. No. 5,565,512 (Saito et al.)). In addition, ammonia-generating compounds may be useful. “Ammonia-generating compounds” include compounds that are solid or liquid at ambient conditions but that generate ammonia under conditions of cure. Examples of such compounds include hexamethylenetetramine (urotropin), dicyandiamide, and substituted and unsubstituted triazine derivatives such as those of the formula:

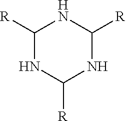

wherein R is a hydrogen atom or a substituted or unsubstituted alkyl, aryl, or aralkyl group having from 1 to about 20 carbon atoms. Specific useful triazine derivatives include hexahydro-1,3,5-s-triazine and acetaldehyde ammonia trimer.

The combination of curative(s) is generally from about 0.01 to about 10 mol % (in some embodiments, from about 0.1 to about 5 mol %) of the total fluoropolymer amount.

Fluoropolymers of the present disclosure made with multifunctional cure site monomers disclosed herein can be used to make cured fluoroelastomers in the form of a variety of articles, including final articles, such as O-rings, and/or preforms from which a final shape is made, (e.g. a tube from which a ring is cut). To form an article, the curable composition can be extruded using a screw type extruder or a piston extruder. The extruded or pre-formed curable compositions can be cured in an oven at ambient pressure. Alternatively, the curable composition can be shaped into an article using injection molding, transfer molding, or compression molding. Injection molding of the curable composition, for example, can be carried out by masticating the curable composition in an extruder screw and collecting it in a heated chamber from which it is injected into a hollow mold cavity by means of a hydraulic piston. After vulcanization the article can then be demolded. The curable composition according to the present disclosure can also be used to prepare cure-in-place gaskets (CIPG) or form-in-place gaskets (FIPG). A bead or thread of the curable composition can be deposited from a nozzle onto a substrate's surface. After forming to a desired gasket pattern, the curable composition may be cured in place with heat, for example, in an oven at ambient pressure. The curable composition according to the present disclosure can also be useful as a fluoroelastomer caulk, which can be useful, for example, to fill voids in, coat, adhere to, seal, and protect various substrates from chemical permeation, corrosion, and abrasion, for example. Fluoroelastomer caulk can be useful as a joint sealant for steel or concrete containers, seals for flue duct expansion joints, door gaskets sealants for industrial ovens, fuel cell sealants or gaskets, and adhesives for bonding fluoroelastomer gaskets (e.g., to metal). In some embodiments, the curable composition can be dispensed by hand and cured with heat at ambient pressure.

Multifunctional compounds of the present disclosure and/or made by the process of the present disclosure wherein at least one of X or Y is —C(O)NR¹—SO₂—R_{f}¹—SO₂Z as described above in any of their embodiments can be copolymerized with compounds represented by formula R^{a}CF═CR^{a}₂, as described above in any of their embodiments. In some embodiments, the compound represented by formula R^{a}CF═CR^{a}₂ is TFE. Suitable monomers that may be included in the fourth components to be polymerized can also include compounds represented by formula CF₂═CF(CF₂)ₘ(OCₙF₂ₙ)_{z}OR_{f}², as described above in any of their embodiments. The allyl and vinyl ethers represented by formula CF₂═CF(CF₂)ₘ(OCₙF₂ₙ)_{z}OR_{f}² may be present in the fourth components to be polymerized in any useful amount, in some embodiments, in an amount of up to 20, 15, 10, 7.5, or 5 mole percent, based on the total amount of polymerizable components. Conveniently, the reaction can be carried out when Z is F, optionally followed by hydrolysis and/or amination to provide compounds wherein Z is —OM or —NR¹₂ as described above in any of their embodiments.

The copolymer made from multifunctional compounds wherein at least one of X or Y is —C(O)NR¹—SO₂—R_{f}¹—SO₂Z are referred to as ionomers and can have —SO₂Z equivalent weight of up to 1000, 900, 800, 750, 700, or 600. In some embodiments, the copolymer or ionomer has an —SO₂Z equivalent weight of at least 400 or 500. In general, the —SO₂Z equivalent weight of the copolymer refers to the weight of the copolymer containing one mole of —SO₂Z groups, wherein Z is as defined above in any of its embodiments. In some embodiments, the —SO₂Z equivalent weight of the copolymer refers to the weight of the copolymer that will neutralize one equivalent of base. In some embodiments, the —SO₂Z equivalent weight of the copolymer refers to the weight of the copolymer containing one mole of sulfonate groups (i.e., —SO₃⁻). Decreasing the —SO₂Z equivalent weight of the copolymer or ionomer tends to increase electrical conductivity in the copolymer or ionomer but tends to decrease its crystallinity, which may compromise the mechanical properties of the copolymer. Thus, the —SO₂Z equivalent weight may be selected based on a balance of the requirements for the electrical and mechanical properties of the copolymer or ionomer. In some embodiments, the —SO₂Z equivalent weight of the copolymer refers to the weight of the copolymer containing one mole of sulfonamide groups (i.e., —SO₂NH). Sulfonimide groups (e.g., when X is —NZSO₂(CF₂)₁₋₆SO₂X′ and —NZ[SO₂(CF₂)ₐSO₂NZ]₁₋₁₀SO₂(CF₂)ₐSO₂X′) also function as acid groups that can neutralize base as described in further detail below. The effective equivalent weight of copolymers including these groups can be much lower than 1000. Advantageously, when multifunctional compounds in which each X and Y is independently —C(O)NR¹—SO₂—R_{f1}—SO₂Z are copolymerized compounds represented by formula R^{a}CF═CR^{a}₂, the —SO₂Z equivalent weight of the ionomer can be lowered without lowering the molecular weight of the ionomer.

In some embodiments, ionomers are prepared from components including up to 40 mole percent of at least one multifunctional compound in which at least one of X or Y is —C(O)NR¹—SO₂—R_{f}¹—SO₂Z, in any of its embodiments described above, based on the total amount of components. In some embodiments, the components comprise up to 35, 30, 25, or 20 mole percent of a multifunctional compound in which at least one of X or Y is —C(O)NR¹—SO₂—R_{f}¹—SO₂Z, based on the total amount of components. Ionomers may be useful, for example, in the manufacture of polymer electrolyte membranes for use in fuel cells or other electrolytic cells. Ionomers may be useful, for example, in the manufacture of catalyst inks for use in fuel cells.

A membrane electrode assembly (MEA) is the central element of a proton exchange membrane fuel cell, such as a hydrogen fuel cell. Fuel cells are electrochemical cells which produce usable electricity by the catalyzed combination of a fuel such as hydrogen and an oxidant such as oxygen. Typical MEA's comprise a polymer electrolyte membrane (PEM) (also known as an ion conductive membrane (ICM)), which functions as a solid electrolyte. One face of the PEM is in contact with an anode electrode layer and the opposite face is in contact with a cathode electrode layer. Each electrode layer includes electrochemical catalysts, typically including platinum metal. Gas diffusion layers (GDL's) facilitate gas transport to and from the anode and cathode electrode materials and conduct electrical current. The GDL may also be called a fluid transport layer (FTL) or a diffuser/current collector (DCC). The anode and cathode electrode layers may be applied to GDL's in the form of a catalyst ink, and the resulting coated GDL's sandwiched with a PEM to form a five-layer MEA. Alternately, the anode and cathode electrode layers may be applied to opposite sides of the PEM in the form of a catalyst ink, and the resulting catalyst-coated membrane (CCM) sandwiched with two GDL's to form a five-layer MEA. Details concerning the preparation of catalyst inks and their use in membrane assemblies can be found, for example, in U.S. Pat. Publ. No. 2004/0107869 (Velamakanni et al.). In atypical PEM fuel cell, protons are formed at the anode via hydrogen oxidation and transported across the PEM to the cathode to react with oxygen, causing electrical current to flow in an external circuit connecting the electrodes. The PEM forms a durable, non-porous, electrically non-conductive mechanical barrier between the reactant gases, yet it also passes H⁺ ions readily.

The ionomer made from multifunctional compounds wherein at least one of X or Y is —C(O)NR¹—SO₂—R_{f}¹—SO₂Z may be useful for making a catalyst ink composition. In some embodiments, the ionomer is combined with catalyst particles (e.g., metal particles or carbon-supported metal particles). A variety of catalysts may be useful. Typically, carbon-supported catalyst particles are used. Typical carbon-supported catalyst particles are 50% to 90% carbon and 10% to 50% catalyst metal by weight, the catalyst metal typically comprising platinum for the cathode and platinum and ruthenium in a weight ratio of 2:1 for the anode. However, other metals may be useful, for example, gold, silver, palladium, iridium, rhodium, ruthenium, iron, cobalt, nickel, chromium, tungsten, manganese, vanadium, and alloys thereof. To make an MEA or CCM, catalyst may be applied to the PEM by any suitable means, including both hand and machine methods, including hand brushing, notch bar coating, fluid bearing die coating, wire-wound rod coating, fluid bearing coating, slot-fed knife coating, three-roll coating, or decal transfer. Coating may be achieved in one application or in multiple applications. The catalyst ink may be applied to a PEM or a GDL directly, or the catalyst ink may be applied to a transfer substrate, dried, and thereafter applied to the PEM or to the FTL as a decal.

Fluoropolymers that include units derived from the multifunctional compounds of the present disclosure and/or made by the process of the present disclosure can be made by free-radical polymerization. Conveniently, in some embodiments, the methods of making the fluoropolymer disclosed herein includes radical aqueous emulsion polymerization using a sequence of steps, which can include polymerization, coagulation, washing, and drying. In some embodiments, an aqueous emulsion polymerization can be carried out continuously under steady-state conditions. For example, an aqueous emulsion of monomers (e.g, including any of those described above), water, emulsifiers, buffers and catalysts can be fed continuously to a stirred reactor under optimum pressure and temperature conditions while the resulting emulsion or suspension is continuously removed. In some embodiments, batch or semibatch polymerization is conducted by feeding the aforementioned ingredients into a stirred reactor and allowing them to react at a set temperature for a specified length of time or by charging ingredients into the reactor and feeding the monomers into the reactor to maintain a constant pressure until a desired amount of polymer is formed. After polymerization, unreacted monomers are removed from the reactor effluent latex by vaporization at reduced pressure. The fluoropolymer can be recovered from the latex by coagulation.

Emulsifiers are often used when carrying out radical aqueous emulsion polymerization. In the past, perfluorinated alkanoic acids were commonly used as emulsifiers. Perfluorinated alkanoic acids represented by formula Rf-(CF₂)ₙ-A, wherein Rf is a perfluorinated alkyl radical that only contains F and C atoms, n is an integer of 5 to 14 and A is an acid anion salt, for example a —COO⁻X wherein X is H⁺, or a cationic salt such as NH₄⁺ or Na⁺ another metal salt, have come under increased scrutiny because of their environmental persistence and bioaccumulation.

Advantageously, compounds of formula I in which at least one of X or Y is —C(O)—O-M, wherein each M is independently a hydrogen atom, a metallic cation, or a quaternary ammonium cation are useful as emulsifiers. At least the RF group, which includes an alkene group, in the compound of formula I allows the emulsifier to be covalently bonded to the prepared fluoropolymer, eliminating any problems associated with the presence of the emulsifier in the fluoropolymer or finished article and eliminating the need to remove the emulsifier. When used as an emulsifier, the compound of formula I is present in a range from about 0.02% to about 3% by weight with respect to the fluoropolymer. Fluoropolymer particles produced with a fluorinated emulsifier typically have an average diameter, as determined by dynamic light scattering techniques, in range of about 10 nanometers (nm) to about 300 nm, and in some embodiments in range of about 50 nm to about 200 nm.

In some embodiments of the method of making a fluoropolymer according to the present disclosure, including embodiments in which the compound of formula I where at least one of X or Y is —C(O)—O-M, wherein each M is independently a hydrogen atom, a metallic cation, or a quaternary ammonium cation, the polymerization can be carried out without adding any perfluorinated alkanoic acids, in particular perfluorinated alkanoic acids with 6 to 14 carbon atoms, and in particular with 8 carbon atoms (perfluorinated octanoic acid (PFOA)) to the reaction mixture. Therefore, their use is avoided. As another advantage, the fluoropolymers made by the methods of the present disclosure may have a very low extractable amount of perfluorinated alkanoic acids, for example amounts of less than 100 ppb based on the weight of C₆-C₁₂, preferably C₆ to C₁₄ perfluorinated alkanoic acids, and may have an amount of extractable octanoic acid (C₈) of less than 50 ppb, preferably less than 30 ppb—based on the weight of the fluoropolymer.

In some embodiments of the method of making the fluoropolymer according to the present disclosure, a water-soluble initiator (e.g., potassium permanganate or a peroxy sulfuric acid salt) can be useful to start the polymerization process. Salts of peroxy sulfuric acid, such as ammonium persulfate or potassium persulfate, can be applied either alone or in the presence of a reducing agent, such as bisulfites or sulfinates (e.g., fluorinated sulfinates disclosed in U.S. Pat. Nos. 5,285,002 and 5,378,782, both to Grootaert) or the sodium salt of hydroxy methane sulfinic acid (sold under the trade designation “RONGALIT”, BASF Chemical Company, New Jersey, USA). The choice of initiator and reducing agent, if present, will affect the end groups of the copolymer. The concentration range for the initiators and reducing agent can vary from 0.010% to 5% by weight based on the aqueous polymerization medium. When salts of peroxy sulfuric acid are used in the presence of a sulfite or bisulfite salt (e.g., sodium sulfite or potassium sulfite), SO₃ radicals are generated during the polymerization process, resulting in —SO₃⁻ end groups. It might be useful to add metal ions to catalyze or accelerate the formation of —SO₃⁻ radicals. By altering the stoichiometry of the sulfite or bisulfite salt versus the peroxy sulfuric acid salt, one can control the amount of —SO₂X end groups.

In some embodiments of the method of making a fluoropolymer according to the present disclosure (e.g., in compounds of formula I in which X and Y are not —C(O)—O-M), perfluorinated or partially fluorinated emulsifiers may be useful. Generally, these fluorinated emulsifiers are present in a range from about 0.02% to about 3% by weight with respect to the fluoropolymer. Polymer particles produced with a fluorinated emulsifier typically have an average diameter, as determined by dynamic light scattering techniques, in range of about 10 nanometers (nm) to about 300 nm, and in some embodiments in range of about 50 nm to about 200 nm. Examples of suitable emulsifiers include perfluorinated and partially fluorinated emulsifier having the formula [R_{f}—O-L-COO—]ᵢXⁱ⁺ wherein L represents a linear partially or fully fluorinated alkylene group or an aliphatic hydrocarbon group, R_{f} represents a linear partially or fully fluorinated aliphatic group or a linear partially or fully fluorinated aliphatic group interrupted with one or more oxygen atoms, X¹⁺ represents a cation having the valence i and i is 1, 2 or 3. (See, e.g., U.S. Pat. No. 7,671,112 to Hintzer et al.). Additional examples of suitable emulsifiers also include perfluorinated polyether emulsifiers having the formula CF₃—(OCF₂)ₓ—O—CF₂—X′, wherein x has a value of 1 to 6 and X′ represents a carboxylic acid group or salt thereof, and the formula CF₃—O—(CF₂)₃—(OCF(CF₃)—CF₂)_{y}—O-L-Y′ wherein y has a value of 0, 1, 2 or 3, L represents a divalent linking group selected from —CF(CF₃)—, —CF₂—, and —CF₂CF₂—, and Y′ represents a carboxylic acid group or salt thereof (See, e.g., U.S. Pat. Publ. No. 2007/0015865 to Hintzer et al.). Other suitable emulsifiers include perfluorinated polyether emulsifiers having the formula R_{f}—O(CF₂CF₂O)ₓCF₂COOA wherein R_{f} is C_{b}F_{(2b+1)}; where b is 1 to 4, A is a hydrogen atom, an alkali metal or NH₄, and x is an integer of from 1 to 3. (See, e.g., U.S. Pat. Publ. No. 2006/0199898 to Funaki et al.). Suitable emulsifiers also include perfluoriuated emulsifiers having the formula F(CF₂)_{b}O(CF₂CF₂O)ₓCF₂COOA wherein A is a hydrogen atom, an alkali metal or NH₄, b is an integer of from 3 to 10, and x is 0 or an integer of from 1 to 3. (See, e.g., U.S. Pat. Publ. No. 2007/0117915 to Funaki et al.). Further suitable emulsifiers include fluorinated polyether emulsifiers as described in U.S. Pat. No. 6,429,258 to Morgan et al. and perfluorinated or partially fluorinated alkoxy acids and salts thereof wherein the perfluoroalkyl component of the perfluoroalkoxy has 4 to 12 carbon atoms, or 7 to 12 carbon atoms. (See, e.g., U.S. Pat. No. 4,621,116 to Morgan). Suitable emulsifiers also include partially fluorinated polyether emulsifiers having the formula [R_{f}—(O)ₜ—CHF—(CF₂)ₓ—COO—]ᵢXⁱ⁺ wherein R_{f} represents a partially or fully fluorinated aliphatic group optionally interrupted with one or more oxygen atoms, t is 0 or 1 and x is 0 or 1, Xⁱ⁺ represents a cation having a valence i and i is 1, 2 or 3. (See, e.g., U.S. Pat. Publ. No. 2007/0142541 to Hintzer et al.). Further suitable emulsifiers include perfluorinated or partially fluorinated ether-containing emulsifiers as described in U.S. Pat. Publ. Nos. 2006/0223924, 2007/0060699, and 2007/0142513 each to Tsuda et al. and 2006/0281946 to Morita et al. Conveniently, in some embodiments, the method of making the fluoropolymer according to the present disclosure may be conducted in the absence of any of these emulsifiers or any combination thereof, for example, using the methods found in U.S. Pat. Publ. No. 2007/0149733 (Otsuka).

If fluorinated emulsifiers are used, the emulsifiers can be removed or recycled from the fluoropolymer latex, if desired, as described in U.S. Pat. No. 5,442,097 to Obermeier et al., U.S. Pat. No. 6,613,941 to Felix et al., U.S. Pat. No. 6,794,550 to Hintzer et al., U.S. Pat. No. 6,706,193 to Burkard et al., and U.S. Pat. No. 7,018,541 to Hintzer et al.

Most of the initiators described above and any emulsifiers that may be used in the polymerization have an optimum pH-range where they show most efficiency. For this reason, buffers may be useful. Buffers include phosphate, acetate, or carbonate (e.g., (NH₄)₂CO₃ or NaHCO₃) buffers or any other acid or base, such as ammonia or alkali-metal hydroxides. The concentration range for the initiators and buffers can vary from 0.01% to 5% by weight based on the aqueous polymerization medium.

Typical chain-transfer agents like H₂, lower alkanes, alcohols, ethers, esters, and methylene fluoride may be useful in the preparation of the copolymer in some embodiments of the method according to the present disclosure. Termination primarily via chain-transfer results in a polydispersity of about 2.5 or less. In some embodiments of the method according to the present disclosure, the polymerization is carried out without any chain-transfer agents. A lower polydispersity can sometimes be achieved in the absence of chain-transfer agents. Recombination typically leads to a polydispersity of about 1.5 for small conversions.

Useful polymerization temperatures can range from 40° C. to 150° C. Typically, polymerization is carried out in a temperature range from 40° C. to 120° C., 70° C. to 100° C., or 80° C. to 90° C. The polymerization pressure is usually in the range of 0.8 MPa to 2.5 MPa, 1 MPa to 2.5 MPa, and in some embodiments is in the range from 1.0 MPa to 2.0 MPa. Fluorinated monomers such as HFP can be precharged and fed into the reactor as described, for example, in Modern Fluoropolymers, ed. John Scheirs, Wiley & Sons, 1997, p. 241. Perfluoroalkoxyalkyl vinyl or allyl ethers represented by formula CF₂═CF(CF₂)ₘ(OCₙF₂ₙ)_{z}OR_{f}², wherein m, n, z, and R_{f}² are as defined above in any of their embodiments, are typically liquids and may be sprayed into the reactor or added directly, vaporized, or atomized.

To coagulate the obtained fluoropolymer latex, any coagulant which is commonly used for coagulation of a fluoropolymer latex may be used, and it may, for example, be a water-soluble salt (e.g., calcium chloride, magnesium chloride, aluminum chloride or aluminum nitrate), an acid (e.g., nitric acid, hydrochloric acid or sulfuric acid), or a water-soluble organic liquid (e.g., alcohol or acetone). The amount of the coagulant to be added may be in a range of 0.001 to 20 parts by mass, for example, in a range of 0.01 to 10 parts by mass per 100 parts by mass of the latex. Alternatively or additionally, the latex may be frozen for coagulation or mechanically coagulated, for example, with a homogenizer as described in U.S. Pat. No. 5,463,021 (Beyer et al.). Alternatively or additionally, the latex may be coagulated by adding polycations. It may also be useful to avoid acids and alkaline earth metal salts as coagulants to avoid metal contaminants. To avoid coagulation altogether and any contaminants from coagulants, spray drying the latex after polymerization and optional ion-exchange purification may be useful to provide solid fluoropolymer.

In some embodiments, the obtained copolymer or ionomer latices are purified by at least one of anion- or cation-exchange processes to remove functional comonomers, anions, and/or cations before coagulation or spray drying (described below). As used herein, the term “purify” refers to at least partially removing impurities, regardless of whether the removal is complete. The obtained copolymer dispersion after aqueous emulsion polymerization and optional ion-exchange purification can be used as is or, if higher solids are desired, can be upconcentrated.

A coagulated fluoropolymer can be collected by filtration and washed with water. The washing water may, for example, be ion-exchanged water, pure water, or ultrapure water. The amount of the washing water may be from 1 to 5 times by mass to the fluoropolymer, whereby the amount of the emulsifier attached to the fluoropolymer can in some cases be sufficiently reduced by one washing.

In some embodiments of the methods of making the fluoropolymer according to the present disclosure, radical polymerization also can be carried out by suspension polymerization. Suspension polymerization will typically produce particle sizes up to several millimeters.

Fluoropolymers obtained by aqueous emulsion polymerization with inorganic initiators (e.g. persulfates, KMnO₄, etc.) typically have a high number of unstable carbon-based end groups (e.g. more than 200 —COOM or —COF end groups per 10⁶ carbon atoms, wherein M is hydrogen, a metal cation, or NH₂). These carbonyl end groups are vulnerable to peroxide radical attacks, which reduce the oxidative stability of the fluoropolymers. The number of unstable end groups can be determined by Fourier-transform infrared spectroscopy.

Post-fluorination with fluorine gas is commonly used to cope with unstable end groups and any concomitant degradation. Post-fluorination of the fluoropolymer can convert —COOH, amide, hydride, —COF, and other nonperfluorinated end groups or —CF═CF₂ to —CF₃ end groups if desired for some applications. The post-fluorination may be carried out in any convenient manner. The post-fluorination can be conveniently carried out with nitrogen/fluorine gas mixtures in ratios of 75-90:25-10 at temperatures between 20° C. and 250° C., in some embodiments in a range of 150° C. to 250° C. or 70° C. to 120° C., and pressures from 100 KPa to 1000 KPa. Reaction times can range from about four hours to about 16 hours. Under these conditions, most unstable carbon-based end groups are removed, whereas any —SO₂X groups mostly survive and are converted to —SO₂F groups. In some embodiments, post-fluorination is not carried out when non-fluorinated monomers described above are used as monomers in the polymerization.

### Some Embodiments of the Disclosure

In a first embodiment, the present disclosure provides a multifunctional compound represented by formula:

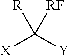

wherein

X and Y are each independently —C(O)—O-M, —C(O)-HAL, —C(O)—NR¹₂, —C≡N, —C(O)NR¹—SO₂—R_{f}¹-W, or a fluorinated alkenyl group that is uninterrupted or interrupted by at least one —O— groups, wherein

each HAL is independently —F, —Cl, or —Br

each R_{f}¹ is independently a fluorinated alkylene group that is uninterrupted or interrupted by at least one —O— group,

each W is independently —F, —SO₂Z, —CF═CF₂, —O—CF═CF₂, or —O—CF₂—CF═CF₂

each Z is independently —F, —Cl, —NR¹₂, or —OM,

each R¹ is independently a hydrogen atom or an alkyl group having up to four carbon atoms, and

each M is independently an alkyl group, a trimethylsilyl group, a hydrogen atom, a metallic cation, or a quaternary ammonium cation;

R is a bromine, chlorine, fluorine, or hydrogen atom; and

RF is a fluorinated alkenyl group that is uninterrupted or interrupted by at least one —O— group and unsubstituted or substituted by at least one chlorine atom, aryl group, or a combination thereof or RF is a fluorinated alkyl group or arylalkylenyl group that is substituted by bromine or iodine and uninterrupted or interrupted by at least one —O— group.

In a second embodiment, the present disclosure provides the multifunctional compound of the first embodiment, wherein R is a fluorine atom.

In a third embodiment, the present disclosure provides the multifunctional compound of the first or second embodiment, wherein RF is a perfluorinated alkenyl group.

In a fourth embodiment, the present disclosure provides the multifunctional compound of any one of the first to third embodiments, wherein RF is —CF═CF₂, —CF═CF—CF₃, or —CF₂—CF═CF₂.

In a fifth embodiment, the present disclosure provides the multifunctional compound of the first or second embodiment, wherein RF is —CCl═CF₂, —CF═CFCl, —CF═CH₂, —CF═CF₂, —CF═CF—CF₃, or —CF₂—CF═CF₂.

In a sixth embodiment, the present disclosure provides the multifunctional compound of any one of the first to fifth embodiments, wherein X and Y are each independently —C(O)—O-M, —C(O)F, —C≡N, or —CF₂—O-perfluorinated alkenyl, and wherein each M is independently an alkyl group, a trimethylsilyl group, a hydrogen atom, a metallic cation, or a quaternary ammonium cation.

In a seventh embodiment, the present disclosure provides the multifunctional compound of any one of the first to sixth embodiments, wherein X and Y are each independently —C(O)—O-M, —C(O)F, —CF₂—O—CF═CF₂, or —CF₂—O—CF₂—CF═CF₂, and wherein each M is independently an alkyl group, a hydrogen atom, a metallic cation, or a quaternary ammonium cation.

In an eighth embodiment, the present disclosure provides the multifunctional compound of any one of the first to fifth embodiments, wherein X and Y are each independently —C(O)NR¹—SO₂—R_{f}¹—SO₂Z, wherein R¹ is hydrogen or methyl, and wherein each R_{f}¹ is independently a perfluorinated alkylene group having up to six carbon atoms.

In a ninth embodiment, the present disclosure provides a fluoropolymer prepared from components comprising the multifunctional compound of any one of the first to eighth embodiments.

In a tenth embodiment, the present disclosure provides a process for making the multifunctional compound of any one of the first to eighth embodiments, the process comprising:

combining first components comprising:
a malonate represented by formula M′O(O)C—C(R)H—C(O)OM′, a base, and a fluorinated compound comprising an alkene group; and
forming a compound represented by formula M′O(O)C—C(R)RF—C(O)OM′,

wherein each M′ is independently an alkyl group or a trimethylsilyl group, R is a fluorine atom or hydrogen atom; and RF is a fluorinated alkenyl group that is uninterrupted or interrupted by at least one —O— group and unsubstituted or substituted by at least one chlorine atom, aryl group, or combination thereof.

In an eleventh embodiment, the present disclosure provides the process of the tenth embodiment, wherein the fluorinated compound comprising the alkene group is R^{a}CF═CR^{a}₂, CF₂═CF—CF₂-LG, or CF₂═CF(CF₂)ₘ(OCₙF₂ₙ)_{z}OR²,

wherein

each R^{a} is independently fluoro, chloro, bromo, hydrogen, a fluoroalkyl group, alkyl having up to 10 carbon atoms, alkoxy having up to 8 carbon atoms, or aryl having up to 8 carbon atoms;

LG is Cl, Br, I, chlorosulfate, fluorosulfate, or trifluoromethyl sulfate;

R_{f}² is a linear or branched perfluoroalkyl group having from 1 to 8 carbon atoms and optionally interrupted by at least one —O— group;

z is 0, 1, or 2;

each n is independently 1, 2, 3, or 4; and

m is 0 or 1.

In a twelfth embodiment, the present disclosure provides the process of the tenth or eleventh embodiments, wherein the fluorinated compound comprising the alkene group is vinylidene fluoride, tetrafluoroethylene, hexafluoropropylene, chlorotrifluoroethylene, or CF₂═CF—CF₂-LG, wherein LG is Cl, Br, I, chlorosulfate, fluorosulfate, or trifluoromethyl sulfate.

In a thirteenth embodiment, the present disclosure provides the process of any one of the tenth to twelfth embodiments, wherein R is a fluorine atom.

In a fourteenth embodiment, the present disclosure provides the process of any one of the tenth to thirteenth embodiment, wherein the base comprises at least one of sodium hydride, sodium bicarbonate, potassium tert-butoxide, cesium carbonate, or n-butyl lithium.

In a fifteenth embodiment, the present disclosure provides the process of any one of the tenth to fourteenth embodiments, further comprising converting the compound represented by formula M′O(O)C—C(R)RF—C(O)OM′ to a compound represented by formula HO(O)C—C(R)RF—C(O)OH, F(O)C—C(R)RF—C(O)F, or R¹₂N(O)C—C(R)RF—C(O)NR¹₂, wherein each R¹ is independently a hydrogen atom or alkyl having up to four carbon atoms.

In a sixteenth embodiment, the present disclosure provides the process of the fifteenth embodiment, further comprising combining second components comprising the compound represented by formula F(O)C—C(R)RF—C(O)F, fluoride ion, and hexafluoropropylene oxide to provide a compound represented by formula:

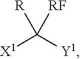

wherein

X¹ is —CF₂—O—CF═CF₂;

Y¹ is —C(O)F or —CF₂—O—CF═CF₂;

R is a fluorine atom or hydrogen atom; and

RF is a fluorinated alkenyl group that is uninterrupted or interrupted by at least one —O— group and substituted or unsubstituted by at least one chlorine atom.

In a seventeenth embodiment, the present disclosure provides the process of the fifteenth embodiment, further comprising combining second components comprising the compound represented by formula F(O)C—C(R)RF—C(O)F, fluoride ion, and CF₂═CF—CF₂-LG, wherein LG is Cl, Br, I, chlorosulfate, fluorosulfate, or trifluoromethyl sulfate, to provide a compound represented by formula:

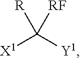

wherein

X¹ is —CF₂—O—CF₂—CF═CF₂;

Y¹ is —C(O)F or —CF₂—O—CF₂—CF═CF₂;

R is a bromine, chlorine, fluorine, or hydrogen atom; and

RF is a fluorinated alkenyl group that is uninterrupted or interrupted by at least one —O— group and unsubstituted or substituted by at least one chlorine atom.

In an eighteenth embodiment, the present disclosure provides the process of the fifteenth embodiment, further comprising combining third components comprising the compound represented by formula R¹₂N(O)C—C(R)RF—C(O)NR¹₂, a base, and a compound represented by formula Hal-SO₂—R_{f}¹-W, wherein each R¹ is independently a hydrogen atom or alkyl having up to four carbon atoms, R_{f}¹ is independently a fluorinated alkylene group that is uninterrupted or interrupted by one or more —O— groups, each W is —F, —SO₂-Hal, —CF═CF₂, —O—CF═CF₂, or —O—CF₂—CF═CF₂, and each Hal is independently —F or —Cl, to provide a compound represented by formula:

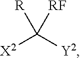

wherein

X² is —C(O)NR¹—SO₂—R_{f}¹-W;

Y² is —C(O)NR¹₂ or —C(O)NR¹—SO₂—R_{f}¹-W;

R is a bromine, chlorine, fluorine, or hydrogen atom; and

RF is a fluorinated alkenyl group that is uninterrupted or interrupted by at least one —O— group and unsubstituted or substituted by at least one chlorine atom.

In a nineteenth embodiment, the present disclosure provides the process of the eighteenth embodiment, wherein W is —SO₂-Hal.

In a twentieth embodiment, the present disclosure provides a method of making a fluoropolymer, the method comprising:

combining fourth components comprising the multifunctional compound of the first to ninth embodiments and at least one fluorinated monomer represented by formula R^{a}CF═CR^{a}₂, CF₂═CF(CF₂)ₘ(OCₙF₂ₙ)_{z}OR_{f}², or a combination thereof, wherein
each R^{a} is independently fluoro, chloro, bromo, hydrogen, a fluoroalkyl group, alkyl having up to 10 carbon atoms, alkoxy having up to 8 carbon atoms, or aryl having up to 8 carbon atoms;
R_{f} is a linear or branched perfluoroalkyl group having from 1 to 8 carbon atoms and optionally interrupted by one or more —O— groups;
z is 0, 1, or 2;
each n is independently 1, 2, 3, or 4; and
and m is 0 or 1; and

copolymerizing the fluorinated monomer and the multifunctional compound.

In a twenty-first embodiment, the present disclosure provides the method of the twentieth embodiment, wherein X and Y are each independently —C≡N or —CF₂—O-perfluorinated alkenyl, the method further comprising crosslinking the fluoropolymer to make a fluoroelastomer.

In a twenty-second embodiment, the present disclosure provides the method of the twentieth embodiment, wherein at least one of X or Y is independently —C(O)—O-M, and wherein each M is independently a hydrogen atom, a metallic cation, or a quaternary ammonium cation, and wherein the multifunctional compound is an emulsifier.

### EXAMPLES

The following specific, but non-limiting, examples will serve to illustrate the present disclosure.

All materials are commercially available, for example from Sigma-Aldrich Chemical Company, Milwaukee, Wis., USA, or known to those skilled in the art, unless otherwise stated or apparent.

The following abbreviations are used in this section: mL=milliliters, g=grams, cm=centimeters, ° C.=degrees Celsius, min=minutes, h=hours, mol=moles, mmol-millimoles, RT=room temperature, mbar=millibar, b.p.=boiling point.

### Example 1 (EX-1) Dimethyl-2-fluor-2-(perfluorprop-1-en-1-yl)malonate

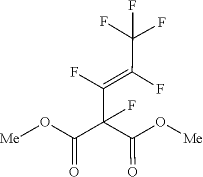 [SMILES: COC(=O)C(F)(C(=O)OC)/C(F)=C(\F)C(F)(F)F]

A three-necked flask (250 mL) equipped with a cooling finger (−50-−60° C.) was charged with dimethyl-2-fluoromalonate (5.3 g, 35 mmol; M=150 g/mol; CAS #344-14-9, available from abcr GmbH, Karlsruhe, Germany) and DMF (80 mL). The obtained solution was cooled to 3-5° C. and by intensive stirring, a suspension of NaH in mineral oil (60%; 1.5 g, 39 mmol) was added. The reaction was kept below 8° C. After the reaction mixture was cooled with liquid nitrogen, HFP (hexafluoropropylene, 5.3 g, 35 mmol) was added. The vessel content was slowly warmed to −10 to −15° C. and was stirred at that temperature for 1.5 h. Then, the reaction mixture was warmed up within 10 h to RT. Afterwards, the reaction mixture was carefully poured on ice-water, washed with a solution of sodium sulfite (40 mL). The raw product was extracted with diethylether (3×400 mL), dried with sodium sulfate. The solvent was evaporated, and the residue was distilled by a Vigreux column. Dimethyl-2-fluor-2-(perfluorprop-1-en-1-yl)malonate was obtained in a yield of 66% (6.5 g, 23 mmol; M=280 g/mol) as clear colorless liquid with a b.p. of 79-81° C. @ 27 mbar.

### Example 2 (EX-2) Diethyl-2-fluoro-2-(perfluorprop-1-en-1-yl)malonate

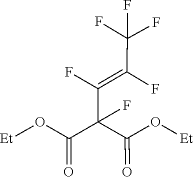 [SMILES: CCOC(=O)C(F)(C(=O)OCC)/C(F)=C(\F)C(F)(F)F]

EX-2 was prepared analogously to the preparation described for EX-1 with the exception that diethyl-2-fluoromalonate (available from abcr GmbH) was used in the place of dimethyl-2-fluoromalonate. Diethyl-2-fluoro-2-(perfluorprop-1-en-1-yl)malonate was isolated in 68% yield (42.5 g, 138 mmol; M=308 g/mol) as a colorless liquid with a b.p. of 43 to 44° C. at 0.7 mbar.

### Example 3 (EX-3) 2-Fluoro-2-(perfluorprop-1-en-1-yl)malonic Acid

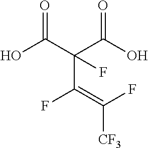 [SMILES: O=C(O)C(F)(C(=O)O)/C(F)=C(\F)C(F)(F)F]

A three-necked flask (25 mL) was charged with a solution of sulfuric acid (98%; 1.4 g, 0.8 mL) in H₂O (12 mL) and cooled to 14° C. Under intensive stirring, dimethyl-2-fluoro-2-(perfluorprop-1-en-1-yl)malonate (1.0 g, 3.6 mmol, which can be prepared as described in EX-1) was added at a rate such that the reaction temperature of 22° C. was not exceeded. The reaction mixture was heated for 17 h at 125° C. After cooling to RT, sodium chloride was added, and the mixture was extracted with methyl-tert-butylether (3×15 mL). The colorless extract was dried over MgSO₄ and the solvent was evaporated in vacuo at 35 to 40° C. The product mixture contained 80 mol % 2-fluoro-2-(perfluoroprop-1-en-1-yl)-malonic acid.

### Example 4 (EX-4) 2-Fluoro-2-(perfluorprop-1-en-1-yl)malonic Acid

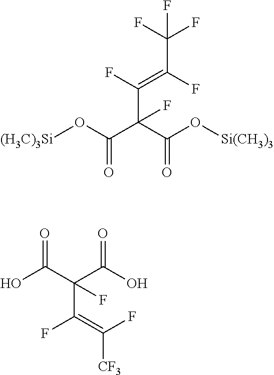

EX-4 was prepared analogously to the preparation described for EX-2 and EX-3 with the exception that Me₃Si—O₂C—CFH—CO₂SiMe₃ was used in place of diethyl-2-fluoromalonate. Me₃Si—O₂C—CFH—CO₂SiMe₃ was prepared from dimethyl-2-fluoromalonate using conventional methods.

### Example 5 (EX-5) 2-Fluoro-2-(perfluoroprop-1-en-1-yl)malonyldichloride

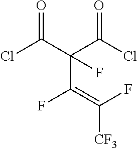 [SMILES: O=C(Cl)C(F)(C(=O)Cl)/C(F)=C(\F)C(F)(F)F]

A three-necked flask (25 mL) was charged with a suspension of PCl₅ (1.5 g, 7.2 mmol; M=208 g/mol) and P(O)Cl₃ (13 mL, 21.8 g, 143 mmol; M=153 g/mol) at RT under inert conditions. Then, carefully 4 to 5 drops of DMF (dimethyl formamide) were added under intensive stirring. The suspension was cooled to 15° C., and under intensive stirring, dimethyl-2-fluoro-2-(perfluoroprop-1-en-1-yl)malonate (which can be prepared as described in EX-1) was added at a rate such that the reaction temperature of 26° C. was not exceeded. The reaction mixture was stirred over night at 100 to 115° C. After cooling down to RT, the product mixture contained 21 mol % 2-fluor-2-(perfluoroprop-1-en-1-yl)malonyldichloride.

### Example 6 (EX-6) Diethyl-2-fluoro-2-(perfluoroallyl)malonate

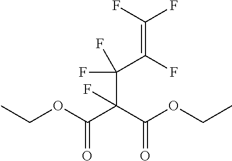 [SMILES: CCOC(=O)C(F)(C(=O)OCC)C(F)(F)C(F)=C(F)F]

A three-necked flask (250 mL), equipped with a thermometer and a reflux condenser, was charged with a suspension of NaH in mineral oil (60%; 2.7 g, 68 mmol) and CH₃CN (90 mL). The suspension was stirred for 20 min at RT, cooled to 8-10° C., and diethyl-2-fluoromalonate (12 g, 67 mmol; M=178 g/mol) was slowly added at a rate such that the reaction temperature of 14° C. was not exceeded. The ice bath was removed, and the vessel content was stirred for 2 h until RT was obtained. Afterwards, the reaction mixture was heated up to 30-35° C. and stirred for 4 h until it became transparent.

This reaction mixture was added via a dropping funnel (100 mL) to a three-necked flask (250 mL), equipped with a thermometer and a reflux condenser, which was charged with a mixture of perfluoroallylfluorosulfate (17 g, 74 mmol; M=230 g/mol; available from Sigma-Aldrich) and acetonitrile (75 mL) at −15-−20° C. After addition, the reaction mixture was slowly warmed up to RT and stirred at that temperature for 18 h. The reaction mixture was concentrated in vacuo and carefully poured at 0° C. into sulfuric acid (2%, 200 mL). The lower phase separated and the water phase was extracted with methyl-tert-butylether (3*100 mL). The organic phases were combined, washed with a saturated solution of NaHCO₃ (3*250 mL) and dried over sodium sulfate. The solvent was evaporated in vacuo and the raw product was distilled with a Vigreux column (20 cm) in vacuo (0.7 mbar). Diethyl-2-fluoro-2-(perfluoroallyl)malonate was isolated in 70% yield (14.4 g, 47 mmol; M=308 g/mol) as a clear yellowish liquid with a b.p. of 56-59° C. at 0.7 mbar.

### Example 7 (EX-7) Diethyl-2-(perfluoroallyl)malonate

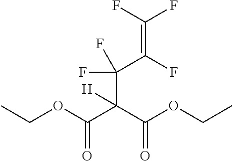 [SMILES: CCOC(=O)C(C(=O)OCC)C(F)(F)C(F)=C(F)F]

A three-necked flask (100 mL), equipped with a thermometer and a reflux condenser, was charged with a suspension of NaH in mineral oil (60%; 2.7 g, 68 mmol) and DMF (50 mL). The suspension was stirred for 15 min at RT, cooled to 6-8° C., and diethylmalonate (10 g, 63 mmol; M=160 g/mol; available from Sigma-Aldrich) was slowly added at a rate such that the reaction temperature of 10° C. was not exceeded. The ice bath was removed, and the vessel content was stirred for 2 h until RT was obtained, and subsequently stirred at RT for 14 h.

The reaction mixture was added via a dropping funnel (100 mL) to a three-necked flask (250 mL), equipped with a thermometer and a reflux condenser, which was charged with a mixture of perfluoroallylfluorosulfate (30 g, 130 mmol; M=230 g/mol) and THF (tetrahydrofuran, 125 mL) at −25-−30° C. After addition, the reaction mixture was slowly warmed up to RT and stirred at that temperature for 16 h. The reaction mixture was concentrated in vacuo and carefully poured at 0° C. into sulfuric acid (2%, 750 mL). The lower phase separated, and the water phase was extracted with methyl-tert-butylether (3*300 mL). The organic phases were combined, washed with cold water (3*350 mL) and dried over magnesium sulfate. The solvent was evaporated in vacuo and the raw product was distilled with a Vigreux column (20 cm) in vacuo (0.7 mbar). Diethyl-2-(perfluoroallyl)malonate was isolated in 37% yield (6.6 g, 23 mmol; M=290 g/mol) as a clear yellowish liquid with a b.p. of 56-59° C. at 0.7 mbar.

Various modifications and alterations of this disclosure may be made by those skilled in the art without departing from the scope and spirit of the disclosure, and it should be understood that this invention is not to be unduly limited to the illustrative embodiments set forth herein.

1. A multifunctional compound represented by formula: 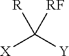
wherein
X and Y are each independently —C(O)—O-M, —C(O)-HAL, —C(O)—NR¹₂, —C≡N, —C(O)NR¹—SO₂—R_{f}¹-W, or a fluorinated alkenyl group that is uninterrupted or interrupted by at least one —O— group, wherein
each HAL is independently —F, —Cl, or —Br,
each R_{f}¹ is independently a fluorinated alkylene group that is uninterrupted or interrupted by at least one —O— group,
each W is independently —F, —SO₂Z, —CF═CF₂, —O—CF═CF₂, or —O—CF₂—CF═CF₂;
each Z is independently —F, —Cl, —NR¹₂, or —OM,
each R¹ is independently a hydrogen atom or alkyl having up to four carbon atoms, and
each M is independently an alkyl group, a trimethylsilyl group, a hydrogen atom, a metallic cation, or a quaternary ammonium cation;
R is a bromine, chlorine, fluorine, or hydrogen atom; and
RF is a fluorinated alkenyl group comprising up to 10 carbon atoms that is uninterrupted or interrupted by at least one —O— group and unsubstituted or substituted by at least one chlorine atom, aryl group, or a combination thereof or RF is a fluorinated alkyl group or arylalkylenyl group that is substituted by bromine or iodine and uninterrupted or interrupted by at least one —O— group.

2. The multifunctional compound of claim 1, wherein R is a fluorine atom, and RF is a perfluorinated alkenyl group.

3. The multifunctional compound of claim 1, wherein X and Y are each independently
—C(O)—O-M, —C(O)F, —C≡N, or —CF₂—O-perfluorinated alkenyl, and wherein each M is independently an alkyl group, a trimethylsilyl group, a hydrogen atom, a metallic cation, or a quaternary ammonium cation.

4. A fluoropolymer prepared from components comprising the multifunctional compound of claim 1.

5. A process for making a multifunctional compound, the process comprising:
combining first components comprising:
a malonate represented by formula M′O(O)C—C(R)H—C(O)OM′, a base, and a fluorinated compound comprising an alkene group; and
forming a compound represented by formula M′O(O)C—C(R)RF—C(O)OM′,
wherein each M′ is independently an alkyl group or a trimethylsilyl group, R is a bromine, chlorine, fluorine, or hydrogen atom; and RF is fluorinated alkenyl group comprising up to 10 carbon atoms that is uninterrupted or interrupted by at least one —O— group and unsubstituted or substituted by at least one chlorine atom, aryl group, or combination thereof.

6. The process of claim 5, wherein the fluorinated compound comprising the alkene group is R^{a}CF═CR^{a}₂, CF₂═CF—CF₂-LG, or CF₂═CF(CF₂)ₘ(OCₙF₂ₙ)_{z}OR_{f}²,
wherein
each R^{a} is independently fluoro, chloro, bromo, hydrogen, a fluoroalkyl group, alkyl having up to 10 carbon atoms, alkoxy having up to 8 carbon atoms, or aryl having up to 8 carbon atoms;
LG is Cl, Br, I, chlorosulfate, fluorosulfate, or trifluoromethyl sulfate;
R_{f}² is a linear or branched perfluoroalkyl group having from 1 to 8 carbon atoms and optionally interrupted by at least one —O— group;
z is 0, 1, or 2;
each n is independently 1, 2, 3, or 4; and
mis 0 or 1.

7. The process of claim 5, wherein the fluorinated compound comprising the alkene group is vinylidene fluoride, tetrafluoroethylene, hexafluoropropylene, chlorotrifluoroethylene, or
CF₂═CF—CF₂-LG, wherein LG is Cl, Br, I, chlorosulfate, fluorosulfate, or trifluoromethyl sulfate.

8. The process of claim 5, wherein the base comprises at least one of sodium hydride, sodium bicarbonate, potassium tert-butoxide, cesium carbonate, or n-butyl lithium.

9. The process of claim 8, further comprising converting the compound represented by formula M′O(O)C—C(R)RF—C(O)OM′ to a compound represented by formula
HO(O)C—C(R)RF—C(O)OH, F(O)C—C(R)RF—C(O)F, or R¹₂N(O)C—C(R)RF—C(O)NR¹₂, wherein each R¹ is independently a hydrogen atom or alkyl having up to four carbon atoms.

10. The process of claim 9, further comprising combining second components comprising the compound represented by formula F(O)C—C(R)RF—C(O)F, fluoride ion, and hexafluoropropylene oxide, or CF₂═CF—CF₂-LG, wherein LG is Cl, Br, I, chlorosulfate, fluorosulfate, or trifluoromethyl sulfate, to provide a compound represented by formula: 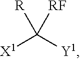
wherein
X¹ is —CF₂—O—CF═CF₂ or —CF₂—O—CF₂—CF═CF₂;
Y¹ is —C(O)F, —CF₂—O—CF═CF₂ or —CF₂—O—CF₂—CF═CF₂;
R is a bromine, chlorine, fluorine, or hydrogen atom; and
RF is fluorinated alkenyl group comprising up to 10 carbon atoms that is uninterrupted or interrupted by at least one —O— group and unsubstituted or substituted by at least one chlorine atom.

11. The process of claim 9, further comprising combining third components comprising the compound represented by formula R¹₂N(O)C—C(R)RF—C(O)NR¹₂, a base, and a compound represented by formula Hal-SO₂—R_{f}¹-W, wherein each R¹ is independently a hydrogen atom or alkyl having up to four carbon atoms, R_{f}¹ is independently a fluorinated alkylene group that is uninterrupted or interrupted by one or more —O— groups, each W is —F, —SO₂-Hal, —CF═CF₂, —O—CF═CF₂, or —O—CF₂—CF═CF₂, and each Hal is independently —F or —Cl, to provide a compound represented by formula: 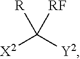
wherein
X² is —C(O)NR¹—SO₂—R_{f}¹-W;
Y² is —C(O)NR¹₂ or —C(O)NR¹—SO₂—R_{f}¹-W;
R is a bromine, chlorine, fluorine, or hydrogen atom; and
RF is fluorinated alkenyl group comprising up to 10 carbon atoms that is uninterrupted or interrupted by at least one —O— group and unsubstituted or substituted by at least one chlorine atom.

12. The process of claim 5, wherein R is fluorine.

13. A method of making a fluoropolymer, the method comprising:
combining fourth components comprising the multifunctional compound of any one of claims 1 to 3 and at least one fluorinated monomer represented by formula R^{a}CF═CR^{a}₂, CF₂═CF(CF₂)ₘ(OCₙF₂ₙ)_{z}OR_{f}², or a combination thereof, wherein
each R^{a} is independently fluoro, chloro, bromo, hydrogen, a fluoroalkyl group, alkyl having up to 10 carbon atoms, alkoxy having up to 8 carbon atoms, or aryl having up to 8 carbon atoms;
R_{f} is a linear or branched perfluoroalkyl group having from 1 to 8 carbon atoms and optionally interrupted by at least one —O— group;
z is 0, 1, or 2;
each n is independently 1, 2, 3, or 4; and
and m is 0 or 1; and
copolymerizing the fluorinated monomer and the multifunctional compound.

14. The method of claim 13, wherein X and Y are each independently —C≡N or —CF₂—O-perfluorinated alkenyl, the method further comprising crosslinking the fluoropolymer to make a fluoroelastomer.

15. The method of claim 13, wherein at least one of X or Y is independently —C(O)—O-M, and wherein each M is independently a hydrogen atom, a metallic cation, or a quaternary ammonium cation, and wherein the multifunctional compound is an emulsifier.

16. The multifunctional compound of claim 1, wherein R is a bromine, chlorine, or fluorine atom.

17. The multifunctional compound of claim 1, wherein X and Y are each independently —C(O)—O-M, —C(O)-HAL, —C(O)—NR¹₂, —C(O)NR¹—SO₂—R_{f}¹-W, or a fluorinated alkenyl group that is uninterrupted or interrupted by at least one —O— group.

18. The multifunctional compound of claim 1, wherein RF is a perfluorinated alkenyl group comprising up to 10 carbon atoms that is uninterrupted or interrupted by at least one —O— group and unsubstituted or substituted by at least one chlorine atom, aryl group, or a combination thereof or RF is a perfluorinated alkyl group or fluorinated arylalkylenyl group that is substituted by bromine or iodine and uninterrupted or interrupted by at least one —O— group.
